(19) United States
(12) Patent Application Publication
(10) Pub. No.: US 2014/0323727 A1
(43) Pub. Date: Oct. 30, 2014

(54) NOVEL PROCESS FOR PREPARING PHENYLCYCLOPROPYLAMINE DERIVATIVES USING NOVEL INTERMEDIATES

(71) Applicant: ACTAVIS GROUP PTC EHF, HAFNARFJORDUR (IS)

(72) Inventors: Anil Shahaji Khile, Navi Mumbai (IN); Jayesh Patel, Palanpur (IN); Nikhil Trivedi, Maharashtra (IN); Nitin Sharadchandra Pradhan, Maharashtra (IN)

(21) Appl. No.: 14/320,853

(22) Filed: Jul. 1, 2014

Related U.S. Application Data
(62) Division of application No. 13/642,239, filed on Nov. 29, 2012.

(30) Foreign Application Priority Data
Apr. 20, 2010 (IN) 1099/CHE/2010
Jan. 6, 2011 (IN) 43/CHE/2011

(51) Int. Cl. C07C 209/34; C07C 205/26; C07C 205/05; C07C 205/45

(52) U.S. Cl. CPC C07C 209/34; C07C 205/45; C07C 205/26; C07C 205/05
USPC 544/254; 564/307; 568/306; 568/705; 568/928

(57) ABSTRACT

Provided herein is a novel process for the preparation of phenylcyclopropylamine derivatives, which are useful intermediates in the preparation of triazolo[4,5-d]pyrimidine compounds. Provided particularly herein is a novel, commercially viable and industrially advantageous process for the preparation of a substantially pure ticagrelor intermediate, trans-(1R,2S)-2-(3,4-difluorophenyl)-cyclopropylamine. The intermediate is useful for preparing ticagrelor, or a pharmaceutically acceptable salt thereof, in high yield and purity.

### CROSS REFERENCE TO RELATED APPLICATIONS

This application is a divisional of U.S. application Ser. No. 13/642,239, which is a 371 of PCT/IB2011/001289, filed Apr. 19, 2011, which claims the benefit of priority to Indian provisional application Nos. 1099/CHE/2010, filed on Apr. 20, 2010; and 43/CHE/2011, filed on Jan. 6, 2011; under the provisions of 35 U.S.C. 119 and the International Convention for the protection of Industrial Property, which are incorporated herein by reference in their entirety.

### FIELD OF THE DISCLOSURE

The present disclosure relates to a novel process for the preparation of phenylcyclopropylamine derivatives, which are useful intermediates in the preparation of triazolo[4,5-d]pyrimidine compounds. The present disclosure particularly relates to a novel, commercially viable and industrially advantageous process for the preparation of a substantially pure ticagrelor intermediate, trans-(1R,2S)-2-(3,4-difluorophenyl)-cyclopropylamine. The intermediate is useful for preparing ticagrelor, or a pharmaceutically acceptable salt thereof, in high yield and purity.

### BACKGROUND

U.S. Pat. Nos. 6,251,910 and 6,525,060 disclose a variety of triazolo[4,5-d]pyrimidine derivatives, processes for their preparation, pharmaceutical compositions comprising the derivatives, and methods of use thereof. These compounds act as P_{2T} (P2Y_{ADP} or P2T_{AC}) receptor antagonists and they are indicated for use in therapy as inhibitors of platelet activation, aggregation and degranulation, promoters of platelet disaggregation, and anti-thrombotic agents. Among them, Ticagrelor, [1S-(1α,2α,3β(1S*,2R*),5β)]-3-[7-[2-(3,4-difluorophenyl)cyclo propyl]amino]-5-(propylthio)-3H-1,2,3-triazolo[4,5-d]pyrimidin-3-yl)-5-(2-hydroxyethoxy)-cyclopentane-1,2-diol, acts as an adenosine uptake inhibitor, a platelet aggregation inhibitor, a P2Y12 purinoceptor antagonist, and a coagulation inhibitor. It is indicated for the treatment of thrombosis, angina, ischemic heart diseases, and coronary artery diseases. Ticagrelor is represented by the following structural formula I:

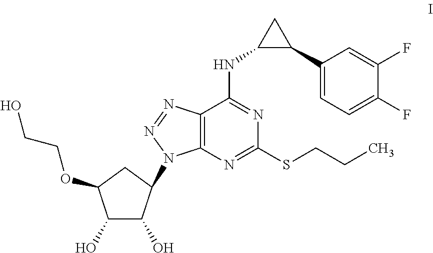 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Ticagrelor is the first reversibly binding oral adenosine diphosphate (ADP) receptor antagonist and is chemically distinct from thienopyridine compounds like clopidogrel. It selectively inhibits P2Y12, a key target receptor for ADP. ADP receptor blockade inhibits the action of platelets in the blood, reducing recurrent thrombotic events. The drug has shown a statistically significant primary efficacy against the widely prescribed clopidogrel (Plavix) in the prevention of cardiovascular (CV) events including myocardial infarction (heart attacks), stroke, and cardiovascular death in patients with acute coronary syndrome (ACS).

Various processes for the preparation of pharmaceutically active triazolo[4,5-d] pyrimidine cyclopentane compounds, preferably ticagrelor, their enantiomers, and their pharmaceutically acceptable salts are disclosed in U.S. Pat. Nos. 6,251,910; 6,525,060; 6,974,868; 7,067,663; 7,122,695 and 7,250,419; U.S. Patent Application Nos. 2007/0265282, 2008/0132719 and 2008/0214812; European Patent Nos. EP0996621 and EP1135391; and PCT Publication Nos. WO2008/018823 and WO2010/030224.

One of the useful intermediates in the synthesis of pharmaceutically active triazolo[4,5-d] pyrimidine cyclopentane compounds is the substituted phenylcyclopropylamine derivative of formula II:

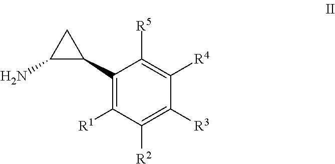

wherein R¹, R², R³, R⁴ and R⁵ are, each independently, selected from hydrogen and a halogen atom, wherein the halogen atom is F, Cl, Br or I; preferably, the halogen atom is F.

In the preparation of ticagrelor, trans-(1R,2S)-2-(3,4-difluorophenyl)-cyclopropylamine of formula IIa:

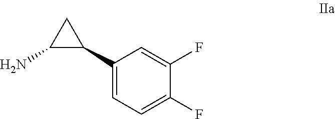 [SMILES: N[C@@H]1C[C@H]1c1ccc(F)c(F)c1]

is a key intermediate.

According to the U.S. Pat. No. 6,251,910 (hereinafter referred to as the '910 patent), the substituted phenylcyclopropylamine derivatives of formula II are prepared by a process as depicted in scheme 1:

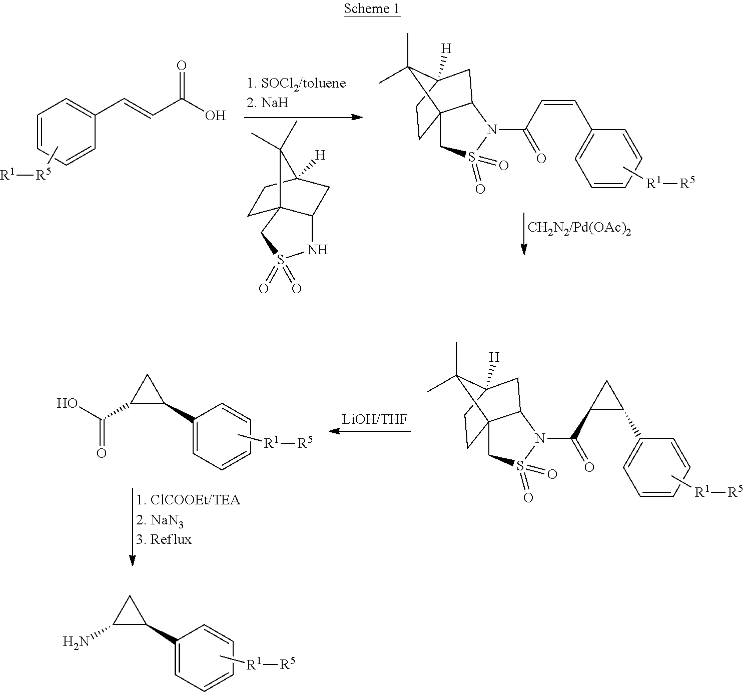

The process for the preparation of substituted phenylcyclopropylamine derivatives disclosed in the '910 patent involves the use of hazardous and explosive materials like sodium hydride, diazomethane and sodium azide. The process also involves the use of highly expensive chiral sultam auxiliary. Moreover, the yields of substituted phenylcyclopropylamine derivatives obtained are low to moderate, and the process involves column chromatographic purifications.

Methods involving column chromatographic purifications are generally undesirable for large-scale operations, thereby making the process commercially unfeasible. The use of explosive reagents like sodium hydride, diazomethane and sodium azide is not advisable, due to the handling difficulties, for scale up operations.

U.S. Pat. No. 7,122,695 (hereinafter referred to as the '695 patent) discloses a process for the preparation of substituted phenylcyclopropylamine derivatives, specifically trans-(1R,2S)-2-(3,4-difluorophenyl)cyclopropylamine and its mandelate salt. The synthesis is depicted in scheme 2:

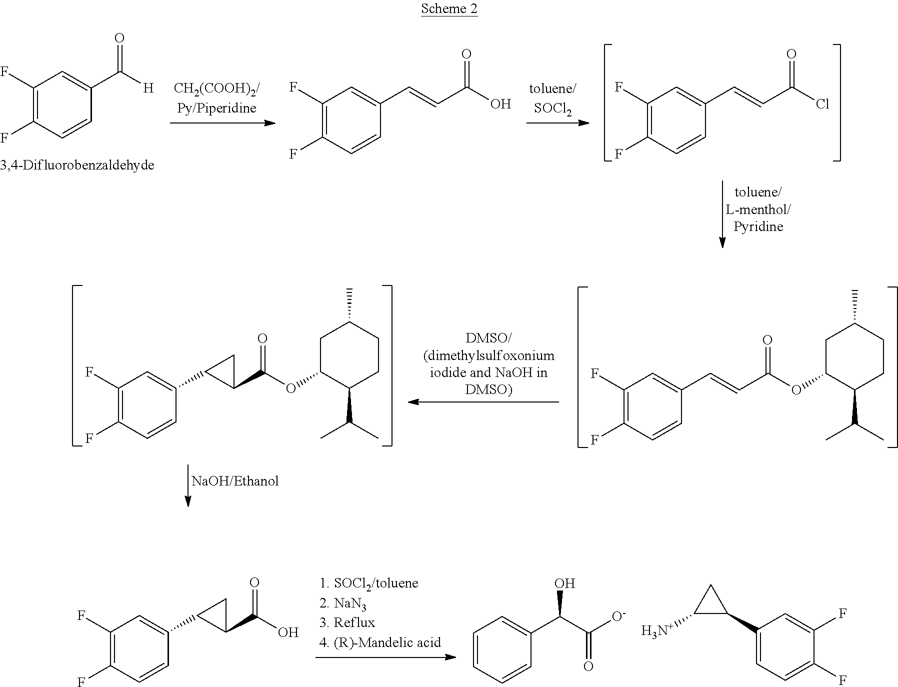

According to the '695 patent, the trans-(1R,2S)-2-(3,4-difluorophenyl)cyclopropylamine is prepared by reacting 3,4-difluorobenzaldehyde with malonic acid in the presence of pyridine and piperidine to produce (E)-3-(3,4-difluorophenyl)-2-propenoic acid, followed by the reaction with thionyl chloride in the presence of pyridine in toluene to produce (E)-3-(3,4-difluorophenyl)-2-propenoyl chloride, which is then reacted with L-menthol in the presence of pyridine in toluene to produce (1R,2S,5R)-2-isopropyl-5-methylcyclohexyl (E)-3-(3,4-difluorophenyl)-2-propenoate. The (1R,2S,5R)-2-isopropyl-5-methylcyclohexyl (E)-3-(3,4-difluorophenyl)-2-propenoate is then reacted with dimethylsulfoxonium methylide in the presence of sodium hydroxide and sodium iodide in dimethylsulfoxide to produce a solution containing (1R,2S,5R)-2-isopropyl-5-methylcyclohexyl trans-2-(3,4-difluorophenyl) cyclopropanecarboxylate, followed by the diastereomeric separation to produce (1R,2S,5R)-2-isopropyl-5-methylcyclohexyl trans-(1R,2R)-2-(3,4-difluorophenyl)cyclopropanecarboxylate. The ester compound is hydrolyzed with sodium hydroxide in ethanol, followed by the acidification with hydrochloric acid to produce trans-(1R,2R)-2-(3,4-difluorophenyl)cyclopropanecarboxylic acid, followed by reaction with thionyl chloride in the presence of pyridine in toluene to produce trans-(1R,2R)-2-(3,4-difluorophenyl) cyclopropanecarbonyl chloride, which is then reacted with sodium azide in the presence of tetrabutylammonium bromide and sodium carbonate in toluene to produce a reaction mass containing trans-(1R,2R)-2-(3,4-difluorophenyl)cyclopropanecarbonyl azide. The azide compound is then added to toluene while stirring at 100° C., followed by acid/base treatment to produce trans-(1R,2R)-2-(3,4-difluorophenyl)cyclopropylamine, which is then converted to its mandelate salt by reaction with R-(−)-mandelic acid in ethyl acetate.

The process disclosed in the '695 patent is lengthy thus resulting in a poor product yield. The process also involves the use of hazardous materials like pyridine and sodium azide.

U.S. Patent Application No. 2008/0132719 (hereinafter referred to as the '719 application) describes a process for the preparation of (1R,2S)-2-(3,4-difluorophenyl)-cyclopropane amine. The synthetic route is depicted in scheme 3:

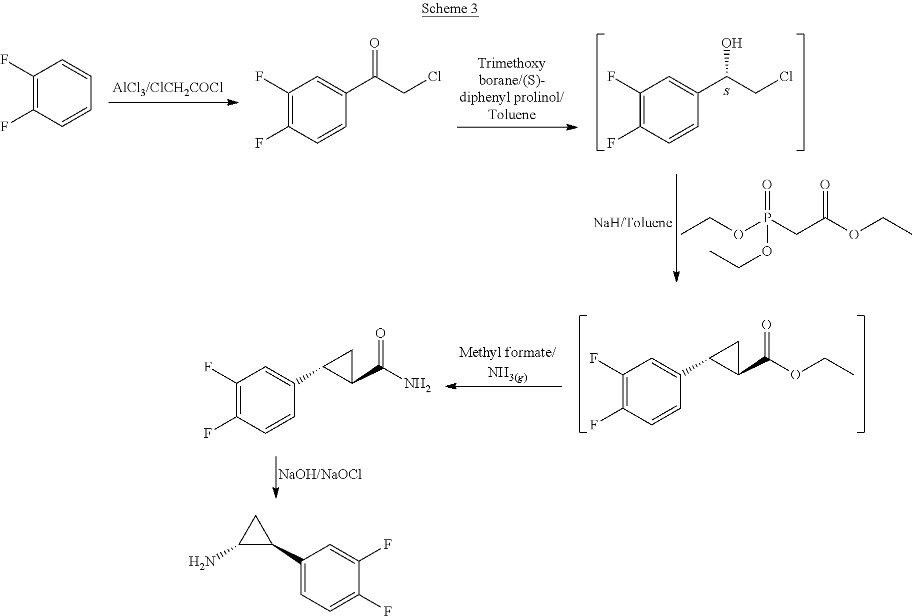

According to the '719 application, the (1R,2S)-2-(3,4-difluorophenyl)-cyclopropane amine is prepared by reacting 1,2-difluorobenzene with chloroacetyl chloride in the presence of aluminium trichloride to produce 2-chloro-1-(3,4-difluorophenyl)ethanone, followed by the reaction with trimethoxy borane and S-diphenylprolinol in toluene to produce 2-chloro-(1S)-(3,4-difluorophenyl)ethanol, which is then reacted with triethyl phosphonoacetate in the presence of sodium hydride in toluene to produce ethyl (1R,2R)-trans-2-(3,4-difluorophenyl)cyclopropyl carboxylate. The ester compound is then reacted with methyl formate in the presence of ammonia to produce (1R,2R)-trans-2-(3,4-difluorophenyl)cyclopropyl carboxamide, which is then reacted with sodium hydroxide and sodium hypochlorite to produce (1R,2S)-2-(3,4-difluorophenyl)-cyclopropane amine.

The process described in the '719 application suffers from the disadvantages since it involves the use of explosive materials like sodium hydride.

PCT Publication No. WO2008/018823 (hereinafter referred to as the '823 publication) describes a process for the preparation of (1R,2S)-2-(3,4-difluorophenyl)-1-cyclopropanamine. The synthetic route is depicted in scheme 4:

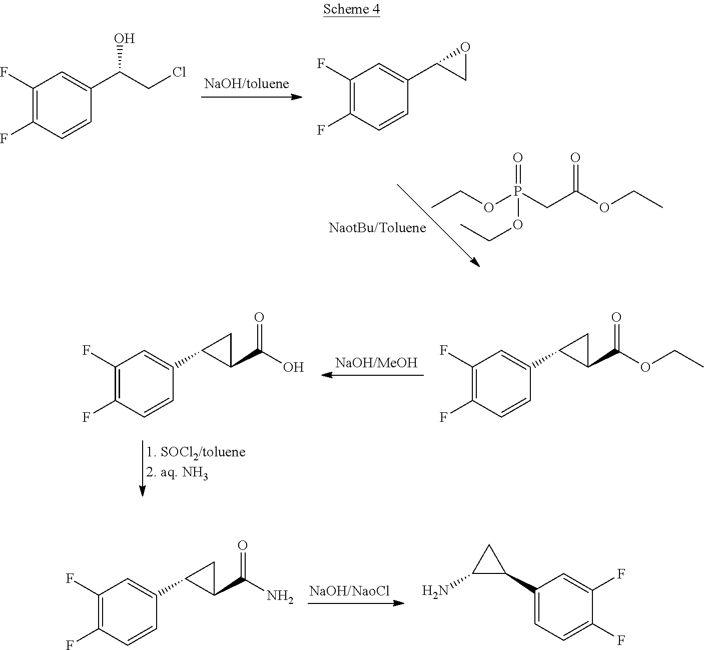

According to the '823 publication, the (1R,2S)-2-(3,4-difluorophenyl)-1-cyclopropanamine is prepared by reacting (1S)-2-chloro-1-(3,4-difluorophenyl)-1-ethanol with sodium hydroxide in toluene to produce (2S)-2-(3,4-difluorophenyl)oxirane, followed by reaction with triethyl phosphonoacetate in the presence of sodium t-butoxide in toluene to produce ethyl (1R,2R)-2-(3,4-difluorophenyl)-1-cyclopropanecarboxylate, which is then hydrolyzed with sodium hydroxide in methanol to produce (1R,2R)-2-(3,4-difluorophenyl)-1-cyclopropanecarboxylic acid. The resulting carboxylic acid compound is reacted with thionyl chloride in toluene to produce a solution of (1R,2R)-2-(3,4-difluorophenyl)-1-cyclopropanecarbonyl chloride, followed by subsequent reaction with aqueous ammonia to produce (1R,2R)-2-(3,4-difluorophenyl)-1-cyclopropanecarboxamide, which is then reacted with sodium hydroxide in the presence of sodium hypochlorite to produce (1R,2S)-2-(3,4-difluorophenyl)-1-cyclopropanamine.

Bioorganic & Medicinal Chemistry, vol. 17(6), pages 2388-2399 (2009) discloses a process for the preparation of racemic trans-2-(3,4-difluorophenyl)cyclopropylamine and its acid addition salt.

J. Med. Chem., vol. 20, No. 7, pages 934-939 (1977) discloses a process for the preparation of 1-aryl-3-nitro-1-propanones from 1-aryl-3-chloro-1-propanones.

J. Org. Chem. 57, pages 3757-3759 (1992) discloses an intramolecular Mitsunobu displacement with carbon nucleophiles for preparation of nitrocyclopropanes from nitroalkanol.

Based on the aforementioned drawbacks, the prior art processes have been found to be unsuitable for the preparation of substituted phenylcyclopropylamine derivatives of formula II at lab scale and in commercial scale operations.

A need remains for an improved and commercially viable process of preparing substituted phenylcyclopropylamine derivatives of formula II with high yields and purity, to resolve the problems associated with the processes described in the prior art, and that will be suitable for large-scale preparation. Desirable process properties include non-hazardous conditions, environmentally friendly and easy to handle reagents, reduced reaction times, reduced cost, greater simplicity, increased purity, and increased yield of the product, thereby enabling the production of triazolo[4,5-d]pyrimidinecyclopentane compounds, preferably ticagrelor, and their pharmaceutically acceptable acid addition salts in high purity and with high yield.

### SUMMARY

In one aspect, provided herein is a novel, efficient, industrially advantageous and environmentally friendly process for the preparation of substituted phenylcyclopropylamine derivatives using novel intermediates, preferably trans-(1R,2S)-2-(3,4-difluorophenyl)-cyclopropylamine or an acid addition salt thereof, in high yield, and with high chemical and enantiomeric purity. Moreover, the process disclosed herein involves non-hazardous and easy to handle reagents, reduced reaction times, and reduced synthesis steps. The process avoids the tedious and cumbersome procedures of the prior processes and is convenient to operate on a commercial scale.

In another aspect, the present disclosure also encompasses the use of pure trans-(1R,2S)-2-(3,4-difluorophenyl)-cyclopropylamine or an acid addition salt thereof obtained by the process disclosed herein for preparing ticagrelor or a pharmaceutically acceptable salt thereof.

The process for the preparation of substituted phenylcyclopropylamine derivatives disclosed herein has the following advantages over the processes described in the prior art:
i) the overall process involves a reduced number of process steps and shorter reaction times;
ii) the process avoids the use of hazardous or explosive chemicals like sodium hydride, diazomethane, pyridine and sodium azide;
iii) the process avoids the use of tedious and cumbersome procedures like column chromatographic purifications and multiple isolations;
iv) the process avoids the use of expensive materials like chiral sultam auxiliary;
v) the process involves easy work-up methods and simple isolation processes, and there is a reduction in chemical waste;
vi) the purity of the product is increased without additional purifications; and
vii) the overall yield of the product is increased.

### DETAILED DESCRIPTION

According to one aspect, there is provided a process for preparing substituted phenylcyclopropylamine derivatives of formula II:

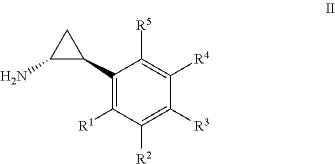

or a stereochemically isomeric form or a mixture of stereochemically isomeric forms thereof, or an acid addition salt thereof; wherein R¹, R², R³, R⁴ and R⁵ are, each independently, selected from hydrogen and a halogen atom, with the proviso that the benzene ring is substituted with at least one or more halogen atoms, wherein the halogen atom is F, Cl, Br or I, preferably, the halogen atom is F; comprising:
a) reacting a halogen substituted phenyl compound of formula VII:

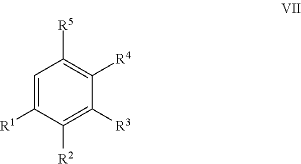

wherein R¹, R², R³, R⁴ and R⁵ are as defined in formula II; with a 3-chloropropionyl halide compound of formula VIII:

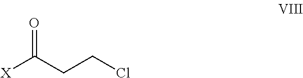

wherein ‘X’ is a leaving group, selected from the group consisting of hydroxy, Cl, Br and I;
in the presence of a Lewis acid in a first solvent to produce an acylated compound of formula VI:

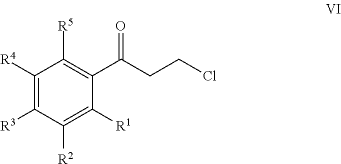

wherein R¹, R², R³, R⁴ and R⁵ are as defined above;
b) nitrating the compound of formula VI with a nitrating agent, in the presence or absence of a metal iodide and an ester suppressant, in a second solvent to produce a substituted 3-nitro-1-propanone compound of formula V:

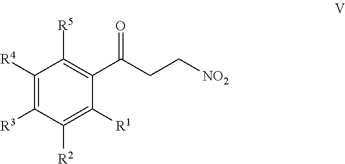

c) subjecting the compound of formula V to asymmetric reduction with a reducing agent in the presence of a chiral auxiliary in a third solvent to produce an optically active substituted 3-nitro-1-propanol compound of formula IV:

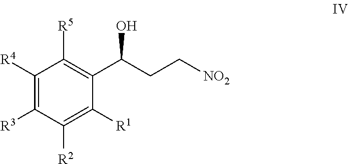

or a stereochemically isomeric form thereof;
d) subjecting the compound of formula IV to intramolecular cyclization in the presence of an azodicarboxylate, optionally in the presence of a phosphine ligand, in a fourth solvent to produce an optically active substituted nitrocyclopropane compound of formula III:

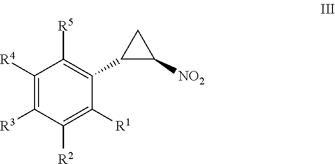

or a stereochemically isomeric form thereof or a mixture of stereochemically isomeric forms thereof; and
e) reducing the substituted nitrocyclopropane compound of formula III with a reducing agent, optionally in the presence of an acid, in a fifth solvent to produce the substituted phenylcyclopropylamine derivatives of formula II or a stereochemically isomeric form or a mixture of stereochemically isomeric forms thereof, and optionally converting the compound of formula II obtained into an acid addition salt thereof.

In one embodiment, the leaving group ‘X’ in the compound of formula VIII is Cl or Br, and more specifically, X is Cl.

In another embodiment, in the compounds of formulae II, III, IV, V, VI and VII, the R¹, R² and R⁵ are H, and wherein the R³ and R⁴ are F.

The compounds of formulae II, III and IV can exist in different isomeric forms such as cis/trans isomers, enantiomers, or diastereomers. The process disclosed herein includes all such isomeric forms and mixtures thereof in all proportions.

In one embodiment, a specific substituted phenylcyclopropylamine derivative of formula II prepared by the process described herein is trans-(1R,2S)-2-(3,4-difluorophenyl)-cyclopropylamine of formula IIa (formula II, wherein R¹, R² and R⁵ are H, and R³ and R⁴ are F):

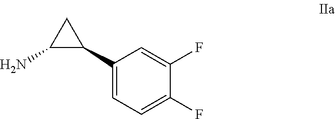 [SMILES: N[C@@H]1C[C@H]1c1ccc(F)c(F)c1]

In another embodiment, a specific substituted phenylcyclopropylamine derivative of formula II prepared by the process described herein is trans-(1S,2R)-2-(3,4-difluorophenyl)-cyclopropylamine of formula IIb (formula II, wherein R¹, R² and R⁵ are H, and R³ and R⁴ are F):

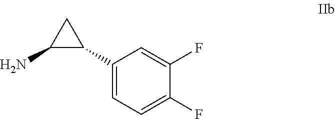 [SMILES: N[C@H]1C[C@@H]1c1ccc(F)c(F)c1]

Exemplary first solvents used in step-(a) include, but are not limited to, an aliphatic or alicyclic hydrocarbon, a chlorinated aliphatic or aromatic hydrocarbon, an aromatic mono or dinitro hydrocarbon, and mixtures thereof. The term solvent also includes mixtures of solvents.

Specifically, the first solvent is selected from the group consisting of n-pentane, n-hexane, n-heptane, cyclohexane, methylene chloride, dichloroethane, chloroform, carbon tetrachloride, dichlorobenzene, nitrobenzene, dinitrobenzene, and mixtures thereof; and a more specific first solvent is dichloromethane or dichlorobenzene.

Exemplary Lewis acid catalysts used in step-(a) include, but are not limited to, aluminium chloride, aluminium bromide, zinc chloride, zinc bromide, boron trifluoride, and mixtures thereof. A specific Lewis acid catalyst is aluminium chloride.

In one embodiment, the acylation reaction in step-(a) is carried out at a temperature of about 0° C. to about 100° C., specifically at a temperature of about 15° C. to about 80° C., and more specifically at a temperature of about 20° C. to about 30° C. The reaction time may vary between about 2 hours to about 40 hours, specifically about 3 hours to about 35 hours, and more specifically about 28 hours to about 32 hours.

It has been surprisingly found that the yield and purity of the acylated compound of formula VI are significantly improved when the acylation reaction is carried out a temperature of about 20° C. to about 30° C. for about 28 hours to about 32 hours.

The reaction mass containing the acylated compound of formula VI obtained in step-(a) may be subjected to usual work up such as a washing, an extraction, a pH adjustment, an evaporation, or a combination thereof. The reaction mass may be used directly in the next step to produce the substituted 3-nitro-1-propanone compound of formula V, or the acylated compound of formula VI may be isolated and then used in the next step.

In one embodiment, the acylated compound of formula VI is isolated from a suitable solvent by conventional methods such as cooling, seeding, partial removal of the solvent from the solution, by adding an anti-solvent to the solution, evaporation, vacuum distillation, or a combination thereof.

The solvent used to isolate the acylated compound of formula VI is selected from the group consisting of water, an aliphatic ether, a hydrocarbon solvent, a chlorinated hydrocarbon, and mixtures thereof. Specifically, the solvent is selected from the group consisting of water, dichloromethane, diethyl ether, diisopropyl ether, n-heptane, n-pentane, n-hexane, cyclohexane, and mixtures thereof. A most specific solvent is dichloromethane.

In another embodiment, the reaction mass containing the acylated compound of formula VI obtained is concentrated and then taken for the next step.

Exemplary second solvents used in step-(b) include, but are not limited to, a ketone, an aliphatic amide, a nitrile, a hydrocarbon, a cyclic ether, an aliphatic ether, a polar aprotic solvent, and mixtures thereof.

In one embodiment, the second solvent is selected from the group consisting of acetone, methyl ethyl ketone, methyl isobutyl ketone, methyl tert-butyl ketone, acetonitrile, tetrahydrofuran, 2-methyl tetrahydrofuran, 1,4-dioxane, diethyl ether, diisopropyl ether, methyl tert-butyl ether, monoglyme, diglyme, n-pentane, n-hexane, n-heptane, cyclohexane, toluene, xylene, N,N-dimethylformamide, N,N-dimethylacetamide, dimethylsulfoxide, N-methylpyrrolidone, and mixtures thereof; and a most specific solvent is N,N-dimethylformamide.

Exemplary nitrating agents used in step-(b) include, but are not limited to, silver nitrite, sodium nitrite, silver chloride and silver nitrate, and mixtures thereof. A most specific nitrating agent is silver nitrite.

Exemplary metal iodides employed for facilitating the nitration reaction in step-(b) include, but are not limited to, potassium iodide, sodium iodide, and the like.

Exemplary ester suppressants employed in the step-(b) include, but are not limited to, benzene-1,3,5-triol (also known as phloroglucinol), and the like.

In one embodiment, the nitration reaction in step-(b) is carried out at a temperature of about 0° C. to about 50° C., specifically at a temperature of about 20° C. to about 40° C., and more specifically at a temperature of about 25° C. to about 35° C. The reaction time may vary between about 30 minutes to about 7 hours, specifically about 1 hour to about 6 hours, and more specifically about 3 hours to about 5 hours. In another embodiment, the reaction mass obtained after completion of the reaction may be quenched in water.

The reaction mass containing the substituted 3-nitro-1-propanone compound of formula V obtained in step-(b) may be subjected to usual work up such as a washing, an extraction, a pH adjustment, an evaporation or a combination thereof. The reaction mass may be used directly in the next step, or the compound of formula V may be isolated, or optionally purified, and then used in the next step.

In one embodiment, the substituted 3-nitro-1-propanone compound of formula V is isolated and/or purified from a suitable solvent by the methods as described above.

The solvent used for isolating or purifying the compound of formula V is selected from the group consisting of an alcohol, a ketone, and mixtures thereof. Specifically, the solvent is selected from the group consisting of methanol, ethanol, isopropyl alcohol, propanol, t-butanol, n-butanol, acetone, methyl ethyl ketone, methyl isobutyl ketone, diethyl ketone, and mixtures thereof; more specifically, the solvent is selected from the group consisting of methanol, ethanol, isopropyl alcohol, acetone, methylethyl ketone, and mixtures thereof; and a most specific solvent is isopropyl alcohol.

Exemplary reducing agents used in step-(c) include, but are not limited to, a borane complex with dimethyl sulfide, N,N-diethylaniline, tetrahydrofuran, picoline, triethylamine, dimethylamine, pyridine, ter-butylamine, 4-methylmorpholine, N-phenyl-morpholine, N-ethyl-N-isopropylaniline, N,N-diisopropylethylamine; L-selectride, (−)-β-Chlorodiisopinocampheyl borane, Rutheneium and Rhodium complexes, and mixtures thereof.

Specifically, the reducing agent is selected from the group consisting of a borane complex with dimethyl sulfide, N,N-diethylaniline, tetrahydrofuran, picoline, triethylamine, dimethylamine, pyridine, ter-butylamine, 4-methylmorpholine, N-phenyl-morpholine, N-ethyl-N-isopropylaniline and N,N-diisopropylethylamine; and a most specific reducing agent is a borane complex with dimethyl sulfide or N,N-diethylaniline.

In addition to the above, other reagents or reagent classes can be used for the same transformation. Particularly preferred methods are based on chiral ruthenium complexes (T. Hamada; T. Torii; K. Izawa; R. Noyori; T. Ikariya, Org. Lett. 2002, 4, 43734376) or chiral chloroborane (J. Chandrasekharan; P. V. Ramachandran; H. C. Brown, J. Org. Chem. 1985, 50, 5448-5450).

Exemplary chiral auxiliaries (or their enantiomers) used in step-(c) are disclosed by, for example, E. J. Corey and C. J. Helal, Angew. Chem. Int. Ed. 1998, 37, 1986-2012; Y. Gao at al., WO 9532937 and Tetrahedron Lett. 1994, 35, 6631-6634; U. Kraatz, DE 3609152; S. Itsuno and K. Ito, J. Org. Chem. 1984, 49, 555-557; G. J. Quallich et al., Tetrahedron Lett. 1993, 34, 41454148; S. Itsuno et al., J. Chem. Soc. Perkin Trans I 1983, 1673-1676; or C. H. Senanayake at al., Tetrahedron Lett. 1998, 39, 1705-1708.

In one embodiment, the chiral auxiliary is selected from the group consisting of (1S,2S)-cis-1-amino-2-indanol, (R) or (S)-2-methyl-CBS-oxazaborolidine, (R) or (S)-o-tolyl-CBS-oxazaborolidine, (R) or (S)-2-(diphenyl hydroxymethyl)pyrrolidine, (1S,2R)-2-amino-1,2-diphenylethanol, (R)-(−)-2-amino-2-phenylethanol, (R)-2-amino-3-methyl-1,1-diphenyl-1-butanol, and (1S,2S)-1-amino-1,2,3,4-tetrahydro-naphthalen-2-ol. A most specific chiral auxiliary is (R) or (S)-2-methyl-CBS-oxazaborolidine.

The generation of the active catalyst may be well performed in situ, as originally described by U. Kraatz in DE 3609152 and by S. Itsuno at al. in J. Chem. Soc. Chem. Commun. 1981, 315-317 and later exemplified by G. J Quallich at al. in Synlett 1993, 929, by combining the chiral auxiliary with excess borane complex in a suitable solvent selected from the group consisting of a chlorinated solvent, an ether, or an aromatic solvent; and a most specific solvent is toluene or tetrahydrofuran.

Exemplary third solvents used in step-(c) include, but are not limited to, a hydrocarbon, a cyclic ether, an aliphatic ether, a chlorinated hydrocarbon and the like, and mixtures thereof.

In one embodiment, the third solvent is selected from the group consisting of tetrahydrofuran, 2-methyl tetrahydrofuran, 1,4-dioxane, diethyl ether, diisopropyl ether, methyl tert-butyl ether, n-pentane, n-hexane, n-heptane, cyclohexane, toluene, xylene, dichloromethane, dichloroethane, chloroform, and mixtures thereof; and most specifically, toluene, dichloromethane, 2-methyl tetrahydrofuran, tetrahydrofuran, and mixtures thereof.

In one embodiment, the reacted stoichiometric ratio of the compound of formula V and the borane is from about 1:0.3 to about 1:2. Specific ratios are 1:0.5; 1:0.6; 1:0.7; 1:0.8; 1:0.9; 1:1; 1:1.2; and 1:1.3.

In another embodiment, the chiral auxiliary is used in an amount of about 1% to about 30% with respect to the compound of formula V, specifically in an amount of about 2% to about 20%, more specifically about 3% to about 10%, and most specifically about 4% to about 8%. In case of effective chiral auxiliaries, the amount of said auxiliary can be consistently lowered, for example, in an amount of about 0.05% to about 2%, and more specifically about 0.5% to about 1%, with respect to the compound of formula V.

In one embodiment, the reaction in step-(c) is carried out at a temperature of about −5° C. to about 80° C., specifically at a temperature of about 10° C. to about 50° C., and most specifically at about 15° C. to about 35° C. In another embodiment, the reaction is carried out for about 1 hour to about 20 hours, specifically for about 3 hours to about 18 hours, and most specifically for about 5 hours to about 15 hours.

It has been observed that, slower addition of the compound of formula V, in the form of a solution in the third solvent, is required to obtain the optically active substituted 3-nitro-1-propanol compound of formula IV with high enantiomeric excess. Specifically, the addition time is between 1 hour 30 minutes and 16 hours, and more specifically between 2 hours and 5 hours.

The reaction mass containing the substituted 3-nitro-1-propanol compound of formula IV obtained in step-(c) may be subjected to usual work up such as a washing, an extraction, a pH adjustment, an evaporation or a combination thereof. The reaction mass may be used directly in the next step, or the compound of formula IV may be isolated, or optionally purified, and then used in the next step.

In one embodiment, the substituted 3-nitro-1-propanol compound of formula IV is isolated and/or purified from a suitable solvent by the methods as described above, wherein the solvent is selected from the group consisting of water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

In another embodiment, the reaction mass obtained after completion of the reaction is followed by the addition of a solvent (e.g., water, methanol, ethanol, or acetone), optionally concentrating the reaction mixture, and then recovering the compound of formula IV by treating with a mixture of aqueous solutions of HCl (preferably a 0.5-1.5 mol/L solution) and an organic solvent (e.g., heptane, ethyl acetate, butyl acetate, methyl t-butyl ether or toluene).

In one embodiment, a specific optically active substituted 3-nitro-1-propanol compound of formula IV prepared by the process described herein is (1S)-1-(3,4-difluorophenyl)-3-nitropropan-1-ol of formula IVa (formula IV, wherein R¹, R² and R⁵ are H, and R³ and R⁴ are F):

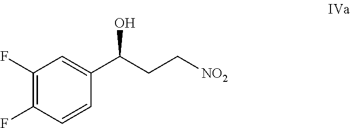 [SMILES: O=[N+]([O-])CC[C@H](O)c1ccc(F)c(F)c1]

In another embodiment, a specific optically active substituted 3-nitro-1-propanol compound of formula IV prepared by the process described herein is (1R)-1-(3,4-difluorophenyl)-3-nitropropan-1-ol of formula IVb (formula IV, wherein R¹, R² and R⁵ are H, and R³ and R⁴ are F):

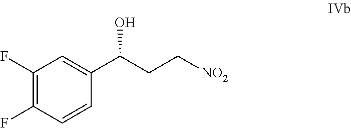 [SMILES: O=[N+]([O-])CC[C@@H](O)c1ccc(F)c(F)c1]

Exemplary fourth solvents used in step-(d) include, but are not limited to, a hydrocarbon, cyclic ethers, an ether, an ester, a nitrile, an aliphatic amide, a chlorinated hydrocarbon, and mixtures thereof.

In one embodiment, the fourth solvent is selected from the group consisting of tetrahydrofuran, 2-methyl tetrahydrofuran, 1,4-dioxane, diethyl ether, diisopropyl ether, methyl tert-butyl ether, dimethoxyethane, diethoxyethane, n-pentane, n-hexane, n-heptane, cyclohexane, toluene, benzene, xylene, dichloromethane, dichloroethane, chloroform, ethyl acetate, isopropyl acetate, tert-butyl acetate, acetonitrile, propionitrile, N,N-dimethylformamamide, N,N-dimethylacetamide, and mixtures thereof; and most specifically benzene, toluene, diochloromethane, 2-methyl tetrahydrofuran, tetrahydrofuran, and mixtures thereof.

Exemplary azodicarboxylates used in step-(d) include, but are not limited to, a di-(C₁₋₄ alkyl)azodicarboxylate, dibenzyl azodicarboxylate and bis-(2,2,2-trichloroethyl)azodicarboxylate. Specific azodicarboxylates are diethyl azodicarboxylate, diisopropyl azodicarboxylate, di-n-propylazodicarboxylate, di-tert-butyl azodicarboxylate and diisobutyl azodicarboxylate; and most specifically diethyl azodicarboxylate or diisopropyl azodicarboxylate.

In another embodiment, the reacted stoichiometric ratio of the compound of formula IV with respect to the dialkylazodicarboxylate is between 1:1 and 1:2. Specific stoichiometric ratios are 1:1.1; 1:1.3; 1:1.5; 1:1.7; and 1:2.

In one embodiment, the reaction in step-(d) is performed in the presence of a phosphine ligand. Exemplary phosphine ligands include, but are not limited to, a trialkylphosphine and a triarylphosphine. Specific phosphine ligands are tributylphosphine, trioctylphosphine, triphenylphosphine and tri (o-tolyl)phosphine; and most specifically triphenylphosphine.

In another embodiment, the reacted stoichiometric ratio of the compound of formula IV with respect to the phosphine ligand is between 1:1 and 1:2. Specific stoichiometric ratios are 1:1.1; 1:1.3; 1:1.5; 1:1.7; and 1:2.

In one embodiment, the reaction in step-(d) is carried out at a temperature of about −5° C. to about 50° C. for at least 30 minutes, specifically at a temperature of about 0° C. to about 30° C. for about 1 hour to about 5 hours, and most specifically at about 0° C. to about 10° C. for about 2 hours to about 3 hours.

If necessary, slower addition of the compound of formula IV or the azodicarboxylate may be required to minimize the impurity formation. The preferred addition time is between 1 hour 30 minutes and 16 hours, and more preferably between 2 hours and 5 hours.

The reaction mass containing the substituted nitrocyclopropane compound of formula III obtained in step-(d) may be subjected to usual work up such as a washing, an extraction, a pH adjustment, an evaporation or a combination thereof. The reaction mass may be used directly in the next step, or the compound of formula III may be isolated, or optionally purified, and then used in the next step.

In one embodiment, the substituted nitrocyclopropane compound of formula III is isolated and/or purified from a suitable solvent by the methods as described above, wherein the solvent is selected from the group consisting of water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

In another embodiment, the reaction mass obtained after completion of the reaction is followed by the addition of a solvent (e.g., water or dilute hydrochloric acid), optionally filtering the reaction mixture, and then recovering the compound of formula III by removal of the solvent.

In one embodiment, a specific optically active substituted nitrocyclopropane compound of formula III prepared by the process described herein is trans-(1R,2S)-2-(3,4-difluorophenyl)-1-nitrocyclopropane of formula Ma (formula III, wherein R¹, R² and R⁵ are H, and R³ and R⁴ are F):

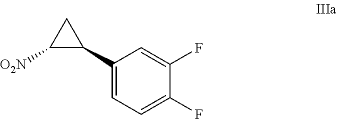 [SMILES: O=[N+]([O-])[C@@H]1C[C@H]1c1ccc(F)c(F)c1]

In another embodiment, a specific optically active substituted nitrocyclopropane compound of formula III prepared by the process described herein is trans-(1S,2R)-2-(3,4-difluorophenyl)-1-nitrocyclopropane of formula IIIb (formula III, wherein R¹, R² and R⁵ are H, and R³ and R⁴ are F):

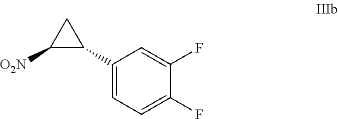 [SMILES: O=[N+]([O-])[C@H]1C[C@@H]1c1ccc(F)c(F)c1]

Exemplary fifth solvents used in step-(e) include, but are not limited to, an alcohol, a hydrocarbon, a cyclic ether, an aliphatic ether, a chlorinated hydrocarbon, and mixtures thereof.

In one embodiment, the fifth solvent is selected from the group consisting of methanol, ethanol, isopropyl alcohol, n-propanol, n-butanol, tetrahydrofuran, 2-methyl tetrahydrofuran, 1,4-dioxane, diethyl ether, diisopropyl ether, methyl tert-butyl ether, dimethoxyethane, diethoxyethane, n-pentane, n-hexane, n-heptane, cyclohexane, toluene, xylene, dichloromethane, dichloroethane, chloroform, and mixtures thereof; and most specifically toluene, diochloromethane, 2-methyl tetrahydrofuran, methanol, ethanol, isopropyl alcohol, tetrahydrofuran, and mixtures thereof.

Exemplary acids used in step-(e) include, but are not limited to, mineral acids and organic acids. In one embodiment, the acid is selected from the group consisting of hydrochloric acid, hydrobromic acid, sulfuric acid, acetic acid, propionic acid, butanoic acid, pentanoic acid, hexanoic acid, and mixtures thereof.

Exemplary reducing agents used in step-(e) include, but are not limited to, noble metal catalysts such as palladium, ruthenium, rhodium, platinum, and their compounds; raney-nickel, ferrous sulfate heptahydrate in aqueous ammonia and the like; and metals such as iron, zinc, cobalt, and mixture thereof.

Alternatively, the reduction can be carried out using other reducing agents which comprise ferric chloride-hydrazine hydrate, sodium dithionite, tin chloride hydrate, tin chloride hydrate-hydrochloric acid, tin-hydrochloric acid, zinc-ammonium formate, zinc-formic acid, zinc-acetic acid, zinc-hydrochloric acid, zinc-hydrazinium monoformate, magnesium-ammonium formate, and mixtures thereof. A specific reducing agent is zinc dust.

In one embodiment, the reaction in step-(e) is carried out at a temperature of about −5° C. to about 80° C. for at least 30 minutes, specifically at a temperature of about 10° C. to about 50° C. for about 1 hour to about 10 hours, and most specifically at about 20° C. to about 40° C. for about 2 hours to about 4 hours.

If necessary, slower addition of the metal catalyst or the acid may be required to minimize the impurity formation. The preferred addition time is between 1 hour 30 minutes and 16 hours, and more preferably between 2 hours and 5 hours.

The reaction mass containing the substituted phenylcyclopropylamine derivatives of formula II or a stereochemically isomeric form or a mixture of stereochemically isomeric forms thereof obtained in step-(e) may be subjected to usual work up, followed by isolating and/or recovering from a suitable solvent by the methods as described above, wherein the solvent is selected from the group consisting of water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

In one embodiment, the substituted phenylcyclopropylamine derivatives of formula II or a stereochemically isomeric form or a mixture of stereochemically isomeric forms thereof obtained in step-(e) is subjected to usual work up and then recovered by techniques such as filtration, filtration under vacuum, decantation, centrifugation, or a combination thereof. In one embodiment, the compound of formula II is recovered by filtration employing a filtration media of, for example, a silica gel or celite.

The use of inexpensive, non-explosive, non-hazardous, readily available and easy to handle reagents and solvents allows the process disclosed herein to be suitable for preparation of the substituted phenylcyclopropylamine derivatives of formula II or a stereochemically isomeric form or a mixture of stereochemically isomeric forms thereof at lab scale and in commercial scale operations.

Acid addition salts of the compounds of formula II can be prepared in high purity by using the substantially pure substituted phenylcyclopropylamine derivatives of formula II or a stereochemically isomeric form or a mixture of stereochemically isomeric forms thereof obtained by the method disclosed herein, by known methods.

In one embodiment, the acid addition salts of the compound of formula II is prepared by the reaction of the compound of formula II with a suitable acid in a suitable solvent, followed by isolating and/or recovering the substantially pure acid addition salt of the compound of formula II.

In another embodiment, the acid addition salts of the compound of formula II in a solid state form are provided. In another embodiment, the acid addition salts of the compound of formula II in a crystalline form are provided. In yet another embodiment, the acid addition salts of the compound of formula II in an amorphous form are provided.

Exemplary solvents used for preparing acid addition salts of the compound of formula II include, but are not limited to, water, an alcohol, a ketone, a chlorinated hydrocarbon, a hydrocarbon, an ester, a nitrile, an ether, a polar aprotic solvent, and mixtures thereof. The term solvent also includes mixtures of solvents.

In one embodiment, the solvent is selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof.

The acid addition salts of substituted phenylcyclopropylamine derivatives of formula II or a stereochemically isomeric form or a mixture of stereochemically isomeric forms thereof are derived from a therapeutically acceptable acid selected from the group consisting of hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, glutaric acid, citraconic acid, glutaconic acid, tartaric acid, mandelic acid, dibenzoyl-L-tartaric acid, di-p-toluoyl-L-tartaric acid, di-p-anisoyl-L-tartaric acid, (R)-(−)-α-methoxyphenyl acetic acid, L-malic acid, (1S)-(+)-10-camphorsulfonic acid, (R) or (S)-α-methoxy-α-(trifluoromethyl)-phenylacetic acid (Mosher's acid), (S) or (R)-(−)-(2-phenylcarbamoyloxy)propionic acid [(S)-(−)-carbamalactic acid], (R) or (S)-para-methylmandelic acid, (R) or (S)-ortho-chloromandelic acid, (R) or (S)-2-hydroxymethylhexanoic acid, (R) or (S)-2-hydroxymethylbutanoic acid, and (R) or (S)-2-hydroxymethylpropanoic acid.

Specific acid addition salts of the compounds of formula II are L-tartrate salt, dibenzoyl-L-tartrate salt, di-p-toluoyl-L-tartrate salt, di-p-anisoyl-L-tartrate, (R)-(−)-mandelate, (R)-(−)-α-methoxyphenyl acetate, L-malate, (1S)-(+)-10-camphorsulfonate, (R) or (S)-α-methoxy-α-(trifluoromethyl)-phenylacetate, (S) or (R)-(−)-(2-phenylcarbamoyloxy)propionate, (R) or (S)-para-methylmandelate, (R) or (S)-ortho-chloromandelate, (R) or (S)-2-hydroxymethylhexanoate, (R) or (S)-2-hydroxymethylbutanoate, and (R) or (S)-2-hydroxymethylpropanoate. More specific acid addition salts of compounds of formula II are L-tartrate salt and (R)-(−)-mandelate salt.

The term “substantially pure substituted phenylcyclopropylamine derivatives” refers to the substituted phenylcyclopropylamine derivatives having a total purity, including both stereochemical and chemical purity, of greater than about 95%, specifically greater than about 98%, more specifically greater than about 99%, and still more specifically greater than about 99.5%. The purity is preferably measured by High Performance Liquid Chromatography (HPLC). For example, the purity of the substituted phenylcyclopropylamine derivatives obtained by the process disclosed herein is about 95% to about 99%, or about 98% to about 99.5%, as measured by HPLC.

According to another aspect, there is provided 1-(3′,4′-difluorophenyl)-3-nitro-propan-1-one of formula Va (formula V, wherein R¹, R² and R⁵ are H, and R³ and R⁴ are F):

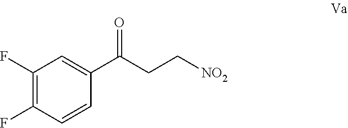 [SMILES: O=C(CC[N+](=O)[O-])c1ccc(F)c(F)c1]

According to another aspect, there is provided an optically active substituted 3-nitro-1-propanol compound of formula IV:

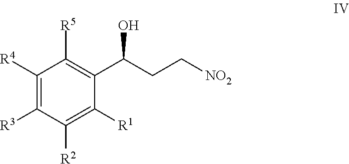

or a stereochemically isomeric form thereof, wherein R¹, R², R³, R⁴ and R⁵ are, each independently, selected from hydrogen and a halogen atom, with the proviso that the benzene ring is substituted with at least one or more halogen atoms, wherein the halogen atom is F, Cl, Br or I; and preferably, the halogen atom is F.

According to another aspect, there is provided an optically active substituted nitrocyclopropane compound of formula III:

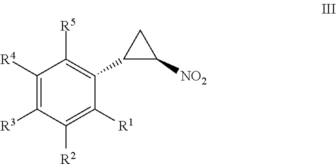

or a stereochemically isomeric form thereof or a mixture of stereochemically isomeric forms thereof, wherein R¹, R², R³, R⁴ and R⁵ are, each independently, selected from hydrogen and a halogen atom, with the proviso that the benzene ring is substituted with at least two or more halogen atoms, wherein the halogen atom is F, Cl, Br or I; and preferably, the halogen atom is F.

Aptly the process for the preparation of the substituted phenylcyclopropylamine derivatives of formula II described herein is adapted to the preparation of triazolo[4,5-d]pyrimidinecyclopentane compounds, preferably ticagrelor of formula I, and their pharmaceutically acceptable acid addition salts, in high enantiomeric and chemical purity.

Ticagrelor and pharmaceutically acceptable acid addition salts of ticagrelor can be prepared in high purity by using the substantially pure trans-(1R,2S)-2-(3,4-difluorophenyl)-cyclopropylamine of formula IIa or an acid addition salt thereof obtained by the methods disclosed herein, by known methods.

The use of the intermediate compounds of formulae III, IV, V and VI, and their stereochemical isomers, in the preparation of substituted phenylcyclopropylamine derivatives of formula II or a stereochemically isomeric form or a mixture of stereochemically isomeric forms thereof is novel and forms further aspect of the present invention.

The following examples are given for the purpose of illustrating the present disclosure and should not be considered as limitation on the scope or spirit of the disclosure.

### EXAMPLES

### Example 1

### Preparation of 3-Chloro-1-(3′,4′-difluorophenyl)-propan-1-one

1,2-Difluorobenzene (225 g, 1.97 mol) was added to a mixture of aluminium chloride (278.82 g, 2.09 mol) and dichloromethane (450 ml) under stirring, followed by slow addition of 3-chloropropionyl chloride (263 g, 2.07 mol) over a period of 1 hour and maintaining the temperature at 25-30° C. The resulting mixtures was heated at reflux (40-45° C.) and maintained for 3 hours. The reaction mass was cooled to 25-30° C., followed by quenching into water (2250 ml) while maintaining the temperature at below 30° C. The resulting mixture was diluted with dichloromethane (1000 ml), followed by stirring for 10 minutes. The resulting layers were separated, the aqueous layer was back extracted with dichloromethane (1000 ml) and then combined with the main organic layer. Saturated sodium bicarbonate solution (600 ml) was added to the combined organic layer, followed by filtration of the biphasic mixture through a hyflo bed. The hyflo bed was washed with dichloromethane (250 ml) and the dichloromethane wash was combined with the main filtrate. The organic layer was separated from the filtrate, followed by washing of the organic layer with water (2×1000 ml). The dichloromethane layer was concentrated under reduced pressure while maintaining the temperature at below 50° C. The concentrated mass was further degassed to obtain 300 g of 3-chloro-1-(3′,4′-difluorophenyl)-propan-1-one as an oil (Yield: 74%).

¹H-NMR (CDCl₃, δ): 3.41 (2H, t), 3.91 (2H, t), 7.29 (1H, m), 7.79 (2H, m).

### Example 2

### Preparation of 3-Chloro-1-(3′,4′-difluorophenyl)-propan-1-one

1,2-Difluorobenzene (1 kg) was added to a mixture of anhydrous aluminium chloride (1.24 kg) and dichloromethane (1.5 L) under stirring at 20-25° C. The container of 1,2-difluorobenzene was flushed with dichloromethane (0.25 L) and then added to the above reaction mass. 3-Chloropropionyl chloride (1.17 kg) was added to the resulting mixture over a period of 60 to 70 minutes while maintaining the temperature at 20-25° C., then the container of 3-chloropropionyl chloride was flushed with dichloromethane (0.25 L) and added to the resulting mass. The resulting mixture was stirred for 30 hours at 20-25° C. The reaction mass obtained after completion of the reaction was quenched into chilled water (10 L) while maintaining the temperature at below 25° C. The resulting mixture was extracted with dichloromethane (2×4 L). The combined dichloromomethane layers were washed with water (2.5 L), followed by washing with 7% aqueous sodium bicarbonate solution (2.5 L) and water (2×2.5 L). The dichloromethane layer was filtered through a hyflo bed and the hyflo bed was washed with dichloromethane (2×1.0 L). The filtrate and washings were combined, followed by concentration under reduced pressure while maintaining the temperature below 50° C. The concentrated mass was further degassed to give 1.584 kg of 3-chloro-1-(3′,4′-difluorophenyl)-propan-1-one as oil (Yield: 88.34%, HPLC Purity: 99.10% by area).

¹H-NMR (CDCl₃, δ): 3.41 (2H, t), 3.91 (2H, t), 7.29 (1H, m), 7.79 (2H, m).

### Example 3

### Preparation of 1-(3′,4′-difluorophenyl)-3-nitro-propan-1-one

Sodium nitrite (168.62 g, 2.44 mol) was added portion wise to a mixture of 3-chloro-1-(3′,4′-difluorophenyl)-propan-1-one (250 g, 1.22 mol), N,N-dimethylformamide (500 ml), phloroglucinol (55 g, 0.436 mol) and sodium iodide (2.5 g) while maintaining the temperature at 25-30° C. The resulting mass was stirred for 3 hours at 25-30° C., followed by quenching into water (2780 ml) while maintaining the temperature at below 5° C. The precipitated product was stirred for 30 minutes at 0-5° C. and the product was isolated by filtration. The wet cake was washed with chilled water (3×450 ml). The wet product was suction dried under reduced pressure and then dissolved in isopropyl alcohol (750 ml) at 50-60° C. The resulting clear solution was gradually cooled to 10-15° C. and maintained for 2 hours. The resulting slurry was cooled further to 0-5° C., followed by stirring for 2 hours at 0-5° C. The product was isolated by filtration and washed with chilled isopropyl alcohol (250 ml), followed by washing of the cake with cyclohexane (2×250 ml). The wet product was dried under reduced pressure at 30-35° C. to constant weight to give 173 g of 1-(3′,4′-difluorophenyl)-3-nitro-propan-1-one (Yield: 65.80%, HPLC Purity: 98.76%).

¹H-NMR (CDCl₃, δ): 3.61 (2H, t), 4.82 (2H, t), 7.29 (1H, m), 7.82 (2H, m).

### Example 4

### Preparation of 1-(3′,4′-difluorophenyl)-3-nitro-propan-1-one

Phloroglucinol (165 g) was added to a mixture of 3-chloro-1-(3′,4′-difluorophenyl)-propan-1-one (750 g), sodium iodide (7.5 g) and N,N-dimethylformamide (750 ml) while maintaining the temperature below 25° C. The resulting mass was cooled to 15-20° C., followed by the addition of sodium nitrite (505.86 g) while maintaining the temperature at below 20° C. The reaction mass temperature was raised to 25-30° C. and maintained for 3 hours. The reaction mass obtained after completion of the reaction was quenched into water (3750 ml) while maintaining the temperature at 20-25° C. The precipitated product was stirred for 60 minutes at 10-15° C. and the product was isolated by filtration. The wet cake was washed with chilled water (2×1500 ml). The wet product was suction dried under reduced pressure and then dissolved in isopropyl alcohol (2250 ml) at 50-60° C. The resulting clear solution was gradually cooled to 10-15° C. and maintained for 2 hours. The resulting slurry was cooled further to 0-5° C., followed by stirring for 2 hours at 0-5° C. The product was isolated by filtration and washed with chilled isopropyl alcohol (187.5 and 750 ml). The wet product was dried under reduced pressure at 30-35° C. till the content of isopropyl alcohol is reached to less than 1000 ppm to give 567 g of 143%4′-difluorophenyl)-3-nitro-propan-1-one (Yield: 71.89%, HPLC Purity: 98.69% by area).

### Example 5

### Preparation of 1-(3′,4′-difluorophenyl)-3-nitro-propan-1-one

3-Chloro-1-(3′,4′-difluorophenyl)-propan-1-one (200 g) and N,N-dimethylformamide (300 ml) were taken into to a reaction assembly under nitrogen atmosphere, followed by cooling the mass to 5-10° C. Phloroglucinol (44 g) and sodium iodide (2.0 g) were added to the resulting suspension while maintaining the temperature at 5-10° C. Sodium nitrite (135 g) was added to the resulting mass while maintaining the temperature at 5-10° C. The resulting reaction mass was stirred for 30 minutes at 5-10° C., followed by raising the temperature of the reaction mass to 25-30° C. and maintaining for 3 to 4 hours. The reaction mass obtained after completion of the reaction was filtered and washed with N,N-dimethylformamide (2×50 ml). The main filtrate and the washing were combined, followed by quenching into water (2500 ml) containing 1-(3′,4′-difluorophenyl)-3-nitro-propan-1-one (2 g, seeding) while maintaining the temperature between 20-25° C. The precipitated product was stirred for 60 minutes at 20-25° C. and the solid was isolated by filtration. The wet cake was washed with water (2×400 ml). The wet product was suction dried under reduced pressure and dissolved in isopropyl alcohol (600 ml) at 50-55° C. The resulting clear solution was gradually cooled to 30-35° C. and then seeded with 1-(3′,4′-difluorophenyl)-3-nitro-propan-1-one (2.0 g) at 30-35° C. The resulting mass obtained after addition of seeding was stirred for 3 hours, followed by cooling to 20-25° C. The resulting slurry was stirred for 10 to 12 hours at 20-25° C. The resulting slurry was further cooled to 0-5° C., followed by stirring for 2 hours at 0-5° C. The product was isolated by filtration and washed with chilled isopropyl alcohol (50 ml+200 ml). The wet product was dried under reduced pressure at 30-35° C. till isopropyl alcohol content is less than 1000 ppm to obtain 148 g of 1-(3′,4′-difluorophenyl)-3-nitro-propan-1-one (Yield: 70.37%, HPLC Purity: 99.78% by area).

### Example 6

### Preparation of 1-(3′,4′-difluorophenyl)-3-nitro-propan-1-one

3-Chloro-1-(3′,4′-difluorophenyl)-propan-1-one (700 g) and N,N-dimethylformamide (1400 ml) were taken into a reaction assembly under nitrogen atmosphere, followed by cooling the mass to 5-10° C. Phloroglucinol (154 g) and sodium iodide (7.0 g) were added to the resulting suspension while maintaining the temperature at 5-10° C. Sodium nitrite (472.5 g) was added to the resulting mass while maintaining the temperature between 5-10° C. The resulting reaction mass was stirred for 30 minutes at 5-10° C., followed by raising the mass temperature to 25-30° C. and maintaining for 3 to 4 hours. Toluene (3500 ml) and water (3500 ml) were added into the reaction mass obtained after completion of the reaction, followed by stirring for 15 minutes. The layers were separated and the aqueous layer was extracted with toluene (2×1750 ml). The toluene layers were combined and washed with water (3×2100 ml). The resulting toluene layer was filtered though hyflo supercel and the bed was washed with toluene (2×350 ml). The main filtrate and the washing were combined and concentrated to dryness while maintaining the temperature at 50° C. under reduced pressure, followed by isopropyl alcohol (2×350 ml) stripping. The concentrated mass was dissolved in isopropyl alcohol (2100 ml) at 50-55° C. The resulting clear solution was gradually cooled to 35-45° C. and then seeded with 1-(3′,4′-difluorophenyl)-3-nitro-propan-1-one (10.0 g) at 35-40° C. The resulting mass obtained after addition of seeding was stirred for 5 hours, followed by cooling to 20-25° C. The resulting slurry was stirred for 8 to 10 hours at 20-25° C. The resulting slurry was further cooled to −5 to 0° C., followed by stirring for 2 hours at −5 to 0° C. The product was isolated by filtration and washed with chilled isopropyl alcohol (175 and 700 ml). The wet product was dried under reduced pressure at 30-35° C. till isopropyl alcohol content is less than 1000 ppm to obtain 575 g of 1-(3′,4′-difluorophenyl)-3-nitro-propan-1-one (Yield: 78.125%, HPLC Purity: 99.86% by area).

### Example 7

### Preparation of (1R)-1-(3,4-difluorophenyl)-3-nitropropan-1-ol using Borane dimethyl sulfide complex

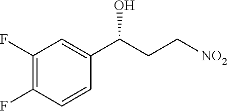 [SMILES: O=[N+]([O-])CC[C@@H](O)c1ccc(F)c(F)c1]

(R)-(+)-2-Methyl-CBS-oxazaborolidine solution (1M in toluene, 2 ml) and tetrahydrofuran (100 ml) were taken into a clean and dry reaction assembly, followed by the addition of borane dimethyl sulfide (30 ml, 0.312 mol) over a period of 15 minutes at 0-5° C. under nitrogen atmosphere. The temperature of the resulting mixture was raised to 25-30° C., followed by stirring for 30 minutes. The resulting reaction mass was followed by the addition of a solution of 1-(3′,4′-difluorophenyl)-3-nitro-propan-1-one (50 g, 0.2324 mol) in tetrahydrofuran (150 ml) over a period of 2 hours at 25-30° C. The resulting reaction mass was stirred for 2 hours. After completion of the reaction, methanol (50 ml) was added to the reaction mass over a period of 30 minutes while maintaining the temperature at below 25° C. The resulting solution was stirred for 30 minutes, followed by distillation of solvent from the reaction mass under reduced pressure at 40-45° C. Dichloromethane (500 ml) and 10% aqueous hydrochloric acid (50 ml) were added to the residue and the resulting acidic solution was stirred for 15 minutes, followed by layer separation. The dichloromethane layer was washed with water (2×300 ml), followed by drying the organic layer over sodium sulfate. The dried organic layer was evaporated under reduced pressure to obtain 50 g of (1R)-1-(3,4-difluorophenyl)-3-nitropropan-1-ol as an oil (Yield: 99.0%).

¹H-NMR (CDCl₃, δ): 2.31 (3H, m), 4.44 (1H, m), 4.59 (1H, m), 4.81 (1H, m), 7.06 (1H, m), 7.15 (2H, m); Mass [M-H]: 215.8; and [R]²⁵_{D}=−27.4° (c 1, CHCl₃).

### Example 8

### Preparation of (1R)-1-(3,4-difluorophenyl)-3-nitropropan-1-ol using Borane dimethyl sulfide complex

(R)-(+)-2-Methyl-CBS-oxazaborolidine solution (1M in toluene, 5 ml) and tetrahydrofuran (225 ml) were taken into a clean and dry reaction assembly, followed by the addition of borane dimethyl sulfide (33.5 ml, 0.3486 mol) over a period of 15 minutes at 25-30° C. under nitrogen atmosphere. The temperature of the resulting mixture was raised to 35-40° C. The resulting mixture was followed by the addition of a solution of 1-(3′,4′-difluorophenyl)-3-nitro-propan-1-one (75 g, 0.3486 mol) in tetrahydrofuran (225 ml) over a period of 3 to 4 hours at 35-40° C. The resulting reaction mass was stirred for 3 hours at 35-40° C. After completion of the reaction, methanol (100 ml) was added to the reaction mass over 30 minutes while maintaining the temperature at below 15° C. The resulting solution was stirred for 30 minutes, followed by the distillation of solvent from the reaction mass under reduced pressure at 40-45° C. Dichloromethane (500 ml) and 10% aqueous hydrochloric acid (500 ml) were added to the residue and the resulting acidic solution was stirred for 15 minutes, followed by layer separation. The dichloromethane layer was washed with water (2×500 ml), followed by drying the organic layer over sodium sulfate. The dried organic layer was evaporated under reduced pressure to obtain 75 g of (1R)-1-(3,4-difluorophenyl)-3-nitropropan-1-ol as an oil (Yield: 99.0%).

### Example 9

### Preparation of (1R)-1-(3,4-difluorophenyl)-3-nitropropan-1-ol using Borane-N,N-diethyl aniline complex

Toluene (90 ml), (R)-(+)-2-Methyl-CBS-oxazaborolidine solution (1M in toluene, 1.4 ml) and borane-N,N-diethyl aniline complex (22.74 g, 0.139 mol) were taken into a clean and dry reaction assembly at 25-30° C. under nitrogen atmosphere. The resulting mixture was stirred for 15 minutes at 25-30° C., followed by heating the mixture at 35-40° C. The resulting mixture was followed by the addition of a solution of 1-(3′,4′-difluorophenyl)-3-nitro-propan-1-one (30 g, 0.139 mol) in toluene (120 ml) over a period of 4 to 6 hours at 35-40° C. The resulting reaction mass was further stirred for 1 hour at 35-40° C. and then cooled to 20-25° C. The resulting mixture was stirred for overnight at 20-25° C. After completion of the reaction, methanol (40 ml) was added to the reaction mass over a period of 30 minutes while maintaining the temperature at below 25° C. The resulting solution was stirred for 30 minutes, followed by the addition of 10% aqueous hydrochloric acid (100 ml). The resulting acidic solution was stirred for 15 minutes and the layers were separated. The aqueous layer was extracted with toluene (100 ml). Both toluene layers were combined and washed with 10% aqueous hydrochloric acid (3×100 ml) and water (2×100 ml). The toluene layer was dried over sodium sulfate to give (1R)-1-(3,4-difluorophenyl)-3-nitropropan-1-ol as an oil.

### Example 10

### Preparation of (1R)-1-(3,4-difluorophenyl)-3-nitropropan-1-ol using Borane-N,N-diethyl aniline complex

Toluene (90 ml), (R)-(+)-2-Methyl-CBS-oxazaborolidine solution (1M in toluene, 1.4 ml) and borane-N,N-diethyl aniline (22.74 g, 0.139 mol) were taken into a clean and dry reaction assembly at 25-30° C. under nitrogen atmosphere. The resulting mixture was stirred for 15 minutes at 25-30° C., followed by heating the mixture at 35-40° C. The resulting mixture was followed by the addition of a solution of 1-(3′,4′-difluorophenyl)-3-nitro-propan-1-one (30 g, 0.139 mol) in toluene (120 ml) over period of 4 to 6 hours at 35-40° C. The resulting reaction mass was further stirred for 1 hour at 35-40° C. and then cooled to 20-25° C. The resulting mixture was stirred for overnight at 20-25° C. After completion of the reaction, methanol (40 ml) was added to the reaction mass over a period of 30 minutes while maintaining the temperature at below 25° C. The resulting solution was stirred for 30 minutes, followed by the addition of 10% aqueous hydrochloric acid (100 ml). The resulting acidic solution was stirred for 15 minutes and the layers were separated. The aqueous layer was extracted with toluene (100 ml). Both toluene layers were combined and washed with 10% aqueous hydrochloric acid (3×100 ml) and water (2×100 ml). The toluene layer was dried over sodium sulfate to give (1R)-1-(3,4-difluorophenyl)-3-nitropropan-1-ol as an oil.

### Example 11

### Preparation of trans-(1S,2R)-2-(3,4-difluorophenyl)-1-nitrocyclopropane

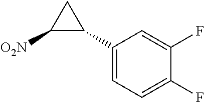 [SMILES: O=[N+]([O-])[C@H]1C[C@@H]1c1ccc(F)c(F)c1]

Triphenylphosphine (136 g, 0.5183 mol) and benzene (400 ml) were taken into a clean and dry reaction assembly, the resulting solution was cooled to 5-10° C., followed by the addition of a solution of diethylazodicarboxylate (90.26 g, 0.5183 mol) in benzene (110 ml) over a period of 30 minutes while maintaining the temperature at 5-10° C. The resulting solution was stirred for 30 minutes, followed by the addition of a solution of (1R)-1-(3,4-difluorophenyl)-3-nitropropan-1-ol (75 g, 0.3455 mol) in benzene (225 ml) over a period of 1 hour while maintaining the temperature between 0-10° C. The resulting reaction mass was stirred for 30 minutes at 0-10° C. After completion of the reaction, the reaction mass was washed with water (2×200 ml), the solvent was removed from the organic layer under reduced pressure and the resulting residue was stirred with 10% ethyl acetate in hexane (1000 ml). The resulting solid was filtered and the filtrate was concentrated under reduced pressure to obtain crude trans-(1S,2R)-2-(3,4-difluorophenyl)-1-nitrocyclopropane (120 g) as an oil.

### Example 12

### Preparation of trans-(1S,2R)-2-(3,4-difluorophenyl)-1-nitrocyclopropane

Triphenyl phosphine (40 g, 0.152 mol) and toluene (90 ml) were taken into a clean and dry reaction assembly, the solution was cooled to 5-10° C., followed by the addition of a solution of diethylazodicarboxylate (26.5 g, 0.152 mol) in toluene (90 ml) over a period of 30 minutes while maintaining the temperature between 5-10° C. The resulting solution was stirred for 30 minutes, followed by the addition of a solution of (1R)-1-(3,4-difluorophenyl)-3-nitropropan-1-ol in toluene (obtained in example 9) over a period of 1 hour while maintaining the temperature between 0-10° C. The resulting reaction mass was stirred for 1 hour at 0-10° C. After completion of the reaction, 10% aqueous hydrochloric acid (100 ml) was added to the reaction mass, followed by filtration of biphasic mixture through a hyflo bed to remove insoluble material. The hyflo bed was washed with toluene (50 ml) and combined the washing with the main toluene filtrate. The aqueous layer was separated from the filtrate, followed by washing the toluene layer with 10% aqueous hydrochloric acid (100 ml) and saturated aqueous sodium chloride solution (100 ml). The toluene was evaporated at 50-55° C. under reduced pressure, followed by purification of residue (silica gel, 1% v/v ethyl acetate in hexane as eluant) to obtain 15.5 g of trans-(1S,2R)-2-(3,4-difluorophenyl)-1-nitrocyclopropane as an oil.

¹H-NMR (CDCl₃, δ): 1.62 (1H, m), 2.24 (1H, m), 3.09 (1H, m), 4.36 (1H, m), 6.89 (2H, m), 7.13 (1H, m); [R]²⁵_{D}=+218.5° (c 1, CHCl₃).

### Example 13

### Preparation of trans-(1S,2R)-2-(3,4-difluorophenyl)-cyclopropylamine

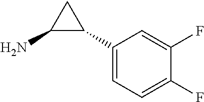 [SMILES: N[C@H]1C[C@@H]1c1ccc(F)c(F)c1]

Crude trans-(1S,2R)-2-(3,4-difluorophenyl)-1-nitrocyclopropane (60 g, 0.2945 mol, obtained in example 11), zinc dust (385 g, 5.8898 mol) and isopropyl alcohol (500 ml) were taken into a clean and dry reaction assembly, followed by the addition of a solution of concentrated hydrochloric acid (307 g, 2.945 ml) diluted in isopropyl alcohol (921 ml) over a period of 1 hour while maintaining the temperature at below 40° C. The reaction mass was stirred for further 1 hour, followed by filtration of the reaction mass through a hyflo bed. The hyflo bed was washed with isopropyl alcohol (2×200 ml) and the isopropyl alcohol filtrate was combined with the main filtrate. The isopropyl alcohol was distilled under reduced pressure and the residue obtained was dissolved in water (1000 ml) and extracted with ethyl acetate (2×500 ml). The ethyl acetate layer was diluted with water (500 ml) and then basified to pH 12 to 13 by the addition of 30% sodium hydroxide solution, followed by filtration of biphasic mixture through a hyflo bed. The hyflo bed was washed with ethyl acetate (100 ml), followed by layer separation. The aqueous layer was extracted with ethyl acetate (250 ml) and the resulting ethyl acetate extract was combined with the main ethyl acetate layer. The combined ethyl acetate layer was washed with water (500 ml) and saturated sodium chloride (500 ml). The ethyl acetate layer was dried over sodium sulfate, followed by evaporation of ethyl acetate under reduced pressure. The resulting residue was dissolved in ethyl acetate (600 ml), followed by the addition of (S)-mandelic acid (44 g). The resulting solution was stirred for 6 hours and the resulting precipitated solid was isolated by filtration. The resulting solid was washed with ethyl acetate (50 ml) and the obtained solid was suspended in ethyl acetate (150 ml), followed by basification to adjust the pH to 12 to 13 using 30% sodium hydroxide solution. The layers were separated and the aqueous layer was extracted with ethyl acetate (100 ml), followed by combining both the ethyl acetate layers. The combined ethyl acetate layer was washed with water (100 ml) and saturated sodium chloride (100 ml). The ethyl acetate layer was dried over sodium sulfate, followed by evaporation of ethyl acetate under reduced pressure to obtain 10 g of trans-(1S,2R)-2-(3,4-difluorophenyl)-cyclopropylamine as an oil.

¹H-NMR (CDCl₃, δ): 0.88 (1H, m), 1.03 (1H, m), 1.71 (2H, bs), 1.79 (1H, m), 2.47 (1H, m), 6.93 (2H, m), 6.97 (1H, m). [R]²⁵_{D}=+79.7° (c 1, CHCl₃).

### Example 14

### Preparation of (1 S)-1-(3,4-difluorophenyl)-3-nitropropan-1-ol

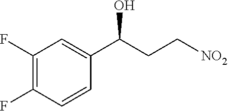 [SMILES: O=[N+]([O-])CC[C@H](O)c1ccc(F)c(F)c1]

Toluene (150 ml), (S)-(−)-2-Methyl-CBS-oxazaborolidine solution (1M in toluene, 7.125 ml) and borane-N,N-diethyl aniline (76 g, 0.4642 mol) were taken into a clean and dry reaction assembly at 25-30° C. under nitrogen atmosphere. The borane-N,N-diethyl aniline container was rinsed with toluene (40 ml) and then transferred into the reaction mass. The resulting mixture was stirred for 10 minutes at 25-30° C., followed by heating the mixture at 35-40° C. The resulting mixture was followed by the addition of a solution of 1-(3′,4′-difluorophenyl)-3-nitro-propan-1-one (95 g, 0.4416 mol) in toluene (285 ml) over a period of 5 to 6 hours at 35-40° C. The resulting reaction mass was further stirred for 1 hour at 35-40° C. and then cooled to 25-30° C. The resulting mixture was stirred overnight at 25-30° C. After completion of the reaction, methanol (47.5 ml) was added to the reaction mass over a period of 30 minutes while maintaining the temperature at below 25° C. The resulting solution was stirred for 30 minutes, followed by the addition of 10% aqueous hydrochloric acid (475 ml). The resulting acidic solution was stirred for 30 minutes, followed by layer separation. The aqueous layer was extracted with toluene (200 ml) and then combined with the main toluene layer. The combined toluene layer was washed with 10% aqueous hydrochloric acid (2×475 ml), water (475 ml), 5% sodium bicarbonate (237.5 ml) and 25% sodium chloride solution (237.5 ml). The toluene layer was dried over sodium sulfate and then concentrated under reduced pressure to obtain 81.5 g of (1S)-1-(3,4-difluorophenyl)-3-nitropropan-1-ol as an oil (Yield: 85%).

¹H-NMR (CDCl₃, δ): 2.24 (3H, m), 4.44 (1H, m), 4.61 (1H, m), 4.82 (1H, m), 7.07 (1H, m), 7.15 (2H, m); [R]²⁵_{D}=+31.3° (c 1, CHC¹₃).

### Example 15

### Preparation of (1 S)-1-(3,4-difluorophenyl)-3-nitropropan-1-ol

Toluene (450 ml), (S)-(−)-2-Methyl-CBS-oxazaborolidine solution (1M in toluene, 22.5 ml) and borane-N,N-diethyl aniline (250.1 g) were taken into a clean and dry reaction assembly at 25-30° C. under nitrogen atmosphere, followed by flushing the assembly with toluene (150 ml). The reaction mass temperature was raised to 35-40° C., followed by the addition of a solution of 1-(3′,4′-difluorophenyl)-3-nitro-propan-1-one (300 g) in toluene (750 ml) over a period of 5 to 6 hours at 35-40° C. The addition funnel was flushed with toluene (150 ml) and then added to the reaction mass. The resulting reaction mass was further stirred for 1 hour at 35-40° C. and then cooled to 25-30° C. The resulting mixture was stirred for 4 hours at 25-30° C. After completion of the reaction, the reaction mass was cooled to 25-30° C., followed by the addition of methanol (150 ml) over a period of 30 minutes, while maintaining the temperature at below 25° C. The resulting solution was stirred for 30 minutes, followed by the addition of dilute aqueous hydrochloric acid (300 ml concentrated hydrochloric acid in 1200 ml of water). The resulting acidic solution was stirred for 15 minutes, followed by the layer separation. The aqueous layer was extracted with toluene (900 ml) and then combined with the main toluene layer. The combined toluene layer was washed twice with dilute aqueous hydrochloric acid (600 ml concentrated hydrochloric acid in 2400 ml of water) and water (2×900 ml). The toluene layer was concentrated under reduced pressure to obtain 291.7 g of (1S)-1-(3,4-difluorophenyl)-3-nitropropan-1-ol as an oil (Yield: 96.27%; HPLC Purity: 98.77% by area; S-isomer: 95.03%; R-isomer: 4.97%; and [R]²⁵_{D}=+39° (c 1, CHCl₃)).

### Example 16

### Preparation of (1S)-1-(3,4-difluorophenyl)-3-nitropropan-1-ol

Toluene (150 ml), (S)-(−)-2-Methyl-CBS-oxazaborolidine solution (1M in toluene, 15 ml) and borane-N,N-diethyl aniline (83.37 g) were taken into a clean and dry reaction assembly at 25-30° C. under nitrogen atmosphere and then flushed the assembly with toluene (50 ml). The reaction mass was stirred for 60 minutes at 25-30° C., followed by the addition of a solution of 1-(3′,4′-difluorophenyl)-3-nitro-propan-1-one (100 g) in toluene (250 ml) over a period of 6 to 7 hours at 25-30° C. The addition funnel was flushed with toluene (50 ml) and then added to the reaction mass. The resulting reaction mass was further stirred for 12 hours at 25-30° C. After completion of the reaction, methanol (50 ml) was added to the reaction mass over a period of 30 minutes while maintaining the temperature below 30° C. The resulting solution was stirred for 30 minutes, followed by the addition of dilute aqueous hydrochloric acid (100 ml concentrated hydrochloric acid in 400 ml of water). The resulting acidic solution was stirred for 15 minutes, followed by the layer separation. The aqueous layer was extracted with toluene (300 ml) and then combined with the main toluene layer. The combined toluene layer was washed twice with dilute aqueous hydrochloric acid (200 ml concentrated hydrochloric acid in 800 ml of water) and water (2×300 ml). The toluene layer was concentrated under reduced pressure to obtain 98 g of (1S)-1-(3,4-difluorophenyl)-3-nitropropan-1-ol as an oil (Yield: 97.0%; HPLC Purity: 97.64% by area; S-isomer: 96.30%; R-isomer: 3.70%; and [R]²⁵_{D}=+37.8° (c 1, CHCl₃)).

### Example 17

### Preparation of (1S)-1-(3,4-difluorophenyl)-3-nitropropan-1-ol

Toluene (150 ml), (S)-(−)-2-Methyl-CBS-oxazaborolidine solution (1M in toluene, 10 ml) and borane-N,N-diethyl aniline (83.37 g) were taken into a clean and dry reaction assembly at 25-30° C. under nitrogen atmosphere and then the assembly was flushed with toluene (50 ml). The reaction mass was stirred for 60 minutes at 25-30° C., followed by the addition of a solution of 1-(3′,4′-difluorophenyl)-3-nitro-propan-1-one (100 g) in toluene (250 ml) over a period of 9 to 10 hours at 25-30° C. The addition funnel was flushed with toluene (50 ml) and then added to the reaction mass. The resulting reaction mass was further stirred for 12 hours at 25-30° C. After completion of the reaction, methanol (50 ml) was added to the reaction mass over a period of 30 minutes while maintaining the temperature below 30° C. The resulting solution was stirred for 30 minutes, followed by the addition of dilute aqueous hydrochloric acid (100 ml concentrated hydrochloric acid in 400 ml of water). The resulting acidic solution was stirred for 15 minutes, followed by layer separation. The aqueous layer was extracted with toluene (300 ml) and then combined with the main toluene layer. The combined toluene layer was washed twice with dilute aqueous hydrochloric acid (200 ml concentrated hydrochloric acid in 800 ml of water) and water (2×300 ml). The toluene layer was concentrated under reduced pressure to obtain 100 g of (1S)-1-(3,4-difluorophenyl)-3-nitropropan-1-ol as an oil (Yield: 99.06%; HPLC Purity: 97.60% by area; S-isomer: 96.32%; R-isomer: 3.68%; and [R]²⁵_{D}=+34.1° (c 1, CHCl₃)).

### Example 18

### Preparation of (1S)-1-(3,4-difluorophenyl)-3-nitropropan-1-ol

Toluene (150 ml), (S)-(−)-2-Methyl-CBS-oxazaborolidine solution (1M in toluene, 10 ml) and borane-N,N-diethyl aniline (83.37 g) were taken into a clean and dry reaction assembly at 15-20° C. under nitrogen atmosphere and the assembly was flushed with toluene (50 ml). The reaction mass was stirred for 90 minutes at 15-20° C., followed by the addition of a solution of 1-(3′,4′-difluorophenyl)-3-nitro-propan-1-one (100 g) in toluene (250 ml) over a period of 9 to 10 hours at 15-20° C. The addition funnel was flushed with toluene (50 ml) and then added to the reaction mass. The resulting reaction mass was further stirred for 12 hours at 15-20° C. After completion of the reaction, methanol (50 ml) was added to the reaction mass over a period of 30 minutes while maintaining the temperature below 30° C. The resulting solution was stirred for 30 minutes, followed by the addition of dilute aqueous hydrochloric acid (100 ml concentrated hydrochloric acid in 400 ml of water). The resulting acidic solution was stirred for 15 minutes, followed by the layer separation. The aqueous layer was extracted with toluene (300 ml) and then combined with the main toluene layer. The combined toluene layer was washed twice with dilute aqueous hydrochloric acid (200 ml concentrated hydrochloric acid in 800 ml of water) and water (2×300 ml). The toluene layer was concentrated under reduced pressure to obtain 97.30 g of (1S)-1-(3,4-difluorophenyl)-3-nitropropan-1-ol as an oil (Yield: 96.4%; HPLC Purity: 97.73% by area; S-isomer: 96.25%; R-isomer-3.75%; and [R]²⁵_{D}=+37.2° (c 1, CHCl₃)).

### Example 19

### Preparation of trans-(1R,2S)-2-(3,4-difluorophenyl)-1-nitrocyclopropane

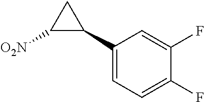

Triphenyl phosphine (33.22 g, 0.1266 mol) and toluene (75 ml) were taken into a clean and dry reaction assembly, the solution was cooled to 5-10° C., followed by the addition of a solution of diisopropylazodicarboxylate (25.6 g, 0.1266 mol) in toluene (75 ml) over a period of 30 minutes while maintaining the temperature at 5-10° C. The resulting solution was stirred for 30 minutes, followed by slow addition of a solution of (1S)-1-(3,4-difluorophenyl)-3-nitropropan-1-ol (25 g, 0.1151 mol) in toluene (75 ml) over a period of 1 hour while maintaining the temperature between 0-10° C. The resulting reaction mass was stirred for 1 hour at 0-10° C. After completion of the reaction, 10% aqueous hydrochloric acid (85 ml) was added to the reaction mass, followed by the filtration of biphasic mixture through a hyflo bed to remove insoluble material. The hyflo bed was washed with toluene (100 ml) and then combined with main toluene filtrate. The aqueous layer was separated from filtrate, followed by washing of toluene layer with 10% aqueous hydrochloric acid (85 ml) and saturated aqueous sodium chloride solution (85 ml). The toluene was evaporated at 50-55° C. under reduced pressure, followed by purification of residue (silica gel, 1% v/v ethyl acetate in hexane as eluant) to obtain 13.3 g of trans-(1R,2S)-2-(3,4-difluorophenyl)-1-nitrocyclopropane as an yellowish oil.

¹H-NMR (CDCl₃, δ): 1.60 (1H, m), 2.21 (1H, m), 3.1 (1H, m), 4.35 (1H, m), 6.89 (2H, m), 7.09 (1H, m); [R]²⁵_{D}=−193.5° (c 1, CHCl₃).

### Example 20

### Preparation of trans-(1R,2S)-2-(3,4-difluorophenyl)-1-nitrocyclopropane

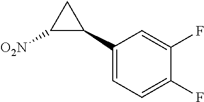

Triphenyl phosphine (415.16 g) and toluene (825 ml) were taken into a clean and dry reaction assembly, the solution was cooled to 5-10° C., followed by the addition of a solution of diisopropylazodicarboxylate (307.15 g) in toluene (700 ml) over a period of 40 minutes while maintaining the temperature between 5-10° C. After completion of addition, the addition funnel was rinsed with 125 ml toluene and then added to the reaction assembly. The resulting solution was stirred for 45 minutes, followed by slow addition of a solution of (1S)-1-(3,4-difluorophenyl)-3-nitropropan-1-ol (275 g) in toluene (700 ml) over a period of 1 hour while maintaining the temperature between 5-10° C. After completion of addition, the addition funnel was rinsed with 125 ml toluene and then added to the reaction assembly. The resulting reaction mass was stirred for 2 hour at 5-10° C. After completion of the reaction, acetic acid (16.5 g) was added to the mass and then stirred for 30 minutes at 5-10° C. The precipitated solid was isolated by filtration and washed with chilled toluene (350 ml). The toluene filtrate and the washing were combined and the solid cake was discarded. The combined toluene filtrate was washed with dilute aqueous hydrochloric acid (137.5 ml of concentrated hydrochloric acid mixed with 825 ml water) and 10% aqueous sodium chloride solution (825 ml). The toluene was evaporated at 50-55° C. under reduced pressure to obtain crude product as dark brown oil. The crude product was further purified by distillation under high vacuum to obtain 250 g of trans-(1R,2S)-2-(3,4-difluorophenyl)-1-nitrocyclopropane as semisolid compound (Yield: 99.2%; HPLC Purity: 89.99%; [R]²⁵_{D}=−191.4° (c 1, CHCl₃)).

### Example 21

### Preparation of trans-(1R,2S)-2-(3,4-difluorophenyl)-cyclopropylamine

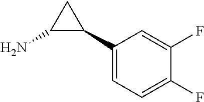

Trans-(1R,2S)-2-(3,4-difluorophenyl)-1-nitrocyclopropane (5 g, 0.0251 mol) and methanol (100 ml) were taken into a reaction flask equipped with a gas sparger, stirrer, and a thermo pocket. The resulting solution was inertized with nitrogen gas, followed by the addition of palladium on carbon (0.5 g, 10%, 50% wet, Type: RD-9210 or RD-841). The resulting suspension was heated at 50-55° C., followed by slow bubbling of hydrogen gas under stirring. Thiophene (0.1 g) was added to the reaction mass, followed by cooling the mass to 25-30° C. The reaction mixture was filtered under nitrogen gas, and the hyflo bed was washed with methanol (2×20 ml). The methanol was evaporated from the filtrate at 50-55° C. under reduced pressure, followed by column purification of residue (silica gel, 5% v/v methanol in dichloromethane as eluent) to obtain 2 g of trans-(1R,2S)-2-(3,4-difluorophenyl)-cyclopropylamine as an yellowish oil.

¹H-NMR (CDCl₃, δ): 0.88 (1H, m), 1.03 (1H, m), 1.71 (2H, bs), 1.79 (1H, m), 2.47 (1H, m), 6.93 (2H, m), 6.97 (1H, m).

### Example 22

### Preparation of trans-(1R,2S)-2-(3,4-difluorophenyl)-cyclopropylamine tartrate salt

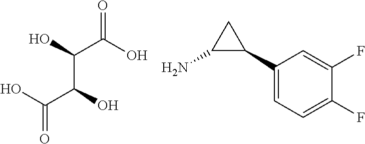

Trans-(1R,2S)-2-(3,4-difluorophenyl)-1-nitrocyclopropane (5.0 g, 0.025 mol), denatured ethanol (50 ml) and 10% hydrochloric acid in denatured ethanol (50 ml) were taken into a clean and dry reaction assembly, followed by cooling the mixture to 5-10° C. Zinc dust (8.17 g, 0.125 mol) was added to the resulting mass over a period of 30 minutes while maintaining the temperature at 15-20° C. The reaction mass was stirred further for 60 minutes, followed by the filtration through a hyflo bed. The hyflo bed was washed with denatured ethanol (2×25 ml) and the washes were combined with the main filtrate. The filtrate was distilled under reduced pressure and the resulting residue was followed by the addition of 10% w/v aqueous sodium hydroxide solution (75.0 ml) and then cooling the mixture to 25 to 30° C. Dichloromethane (50 ml) was added to the cooled mass and stirred for 15 minutes. The resulting suspension was filtered through hyflo bed and washed with dichloromethane (2×25 ml). The layers were separated and the aqueous layer was extracted with dichloromethane (50 ml). The dichloromethane layers were combined and then extracted with 10% aqueous hydrochloric acid (2×25 ml). The resulting aqueous acidic layer was washed with dichloromethane (25 ml). The aqueous acidic layer was basified to pH greater than 11 by adding 10% aqueous sodium hydroxide solution, followed by extraction with dichloromethane (2×50 ml). The resulting dichloromethane layers were combined and then washed with water (2×25 ml). The dichloromethane layer was evaporated under reduced pressure and the resulting oily mass was dissolved in denatured ethanol (15 ml). L-tartaric acid solution (dissolved in 2.48 g in 25 ml denatured ethanol) was slowly added to the clear solution and the resulting slurry was stirred for 1 hour and the solid was recovered by filtration. The resulting solid was washed with denatured ethanol (2×10 ml) and the resulting solid was then suction dried. The wet solid was dried under reduced pressure at 45-50° C. to produce 4.11 g of pure trans-(1R,2S)-2-(3,4-difluorophenyl)-cyclopropylamine tartrate salt as an off white solid.

### Example 23

### Preparation of trans-(1R,2S)-2-(3,4-difluorophenyl)-cyclopropylamine (R)-(−)-mandelate salt

Trans-(1R,2S)-2-(3,4-difluorophenyl)-1-nitrocyclopropane (215.0 g) was added to the pre-cooled methanolic hydrochloric acid (6.0% to 7% w/w HCl, 4300 ml), followed by cooling the mass to −5 to 0° C. Zinc dust (343.71 g) was added to the resulting mass over a period of 2 to 3 hours while maintaining the temperature at −5 to 0° C. The reaction mass was stirred further for 2 hours at −5 to 0° C. After completion of the reaction, the reaction mass was filtered through a hyflo bed and the bed was washed with methanol (2×215 ml). The main filtrate and washings were combined, followed by distillation under reduced pressure. The resulting residue was dissolved in dichloromethane (1075 ml) and then cooled to 10 to 15° C. 25% Aqueous ammonia solution (1290 ml) was added to the cooled solution while maintaining the temperature at below 30° C. The resulting reaction mass was stirred for 15 minutes, followed the by layer separation. The resulting aqueous layer was extracted with dichloromethane (2×537.5 ml) and then combined with the main dichloromethane layer. The combined dichloromethane layer containing the product was extracted thrice with aqueous hydrochloric acid (645 ml of conc. hydrochloric acid mixed with 1935 ml water, 3×865 ml). The aqueous acidic layer containing the product was combined and washed with dichloromethane (645 ml). Dichloromethane (1075 ml) was added to the acidic aqueous layer, followed by the addition of 25% aqueous ammonia solution (1505 ml) while maintaining the temperature at below 30° C. The resulting reaction mass was extracted with dichloromethane (2×645 ml) and then combined with the main dichloromethane layer. The combined dichloromethane layer containing the product was washed with water (645 ml) and evaporated to dryness under reduced pressure. The resulting residue was dissolved in methanol (430 ml), followed slow addition of (R)-(−)-mandelic acid solution (107.5 g in 645 ml methanol) over a period of 40 to 60 minutes while maintaining temperature at 20 to 25° C. The resulting slurry was stirred further for 12 hours at 20 to 25° C., followed by further cooling to 0 to 5° C. The cooled solution was stirred for 2 hours and the solid was isolated by filtration. The resulting solid was washed with chilled methanol (215 ml). The solid was dried under reduced pressure at 40 to 45° C. to obtain 127 g of pure trans-(1R,2S)-2-(3,4-difluorophenyl)-cyclopropylamine (R)-(−)-mandelate salt as a white solid (Yield: 36.61%; HPLC Purity: 99.87% by area; [R]²⁵_{D}=−97.0° (c 1, methanol)).

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

We claim:

1. A process for preparing substituted phenylcyclopropylamine derivatives of formula II: 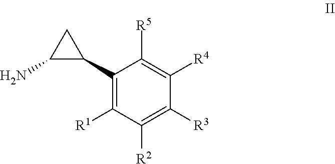
or a stereochemically isomeric form or a mixture of stereochemically isomeric forms thereof, or an acid addition salt thereof; wherein R¹, R², R³, R⁴ and R⁵ are, each independently, selected from hydrogen and a halogen atom, with the proviso that the benzene ring is substituted with at least one or more halogen atoms, wherein the halogen atom is F, Cl, Br or I; comprising:
a) reacting a halogen substituted phenyl compound of formula VII: 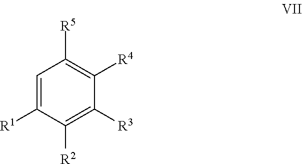
wherein R¹, R², R³, R⁴ and R⁵ are as defined in formula II; with a 3-chloropropionyl halide compound of formula VIII: 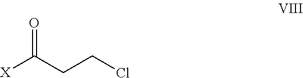
wherein ‘X’ is a leaving group, selected from the group consisting of hydroxy, Cl, Br and I; in the presence of a Lewis acid in a first solvent to produce an acylated compound of formula VI: 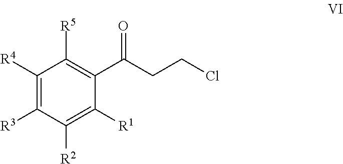
wherein R¹, R², R³, R⁴ and R⁵ are as defined above;
b) nitrating the compound of formula VI with a nitrating agent, in the presence or absence of a metal iodide and an ester suppressant, in a second solvent to produce a substituted 3-nitro-1-propanone compound of formula V: 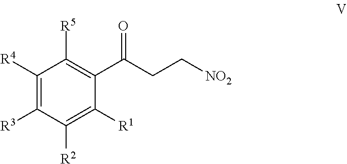
c) subjecting the compound of formula V to asymmetric reduction with a reducing agent in the presence of a chiral auxiliary in a third solvent to produce an optically active substituted 3-nitro-1-propanol compound of formula IV: 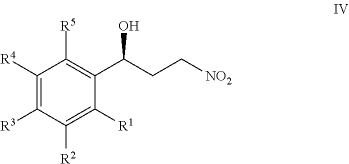
or a stereochemically isomeric form thereof;
d) subjecting the compound of formula IV to intramolecular cyclization in the presence of an azodicarboxylate, optionally in the presence of a phosphine ligand, in a fourth solvent to produce an optically active substituted nitrocyclopropane compound of formula III: 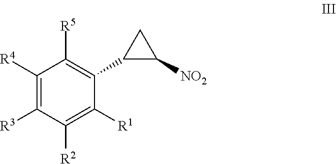
or a stereochemically isomeric form thereof or a mixture of stereochemically isomeric forms thereof; and
e) reducing the substituted nitrocyclopropane compound of formula III with a reducing agent, optionally in the presence of an acid, in a fifth solvent to produce the substituted phenylcyclopropylamine derivatives of formula II or a stereochemically isomeric form or a mixture of stereochemically isomeric forms thereof, and optionally converting the compound of formula II obtained into an acid addition salt thereof.

2. The process of claim 1, wherein the halogen atom in the compounds of formulae II, III, IV, V, VI and VII is F; and wherein the leaving group ‘X’ in the compound of formula VIII is Cl.

3. The process of claim 1, wherein the R¹, R² and R⁵ in the compounds of formulae II, III, IV, V, VI and VII are H, and wherein the R³ and R⁴ are F.

4. The process of claim 1, wherein the first solvent used in step-(a) is selected from the group consisting of an aliphatic or alicyclic hydrocarbon, a chlorinated aliphatic or aromatic hydrocarbon, an aromatic mono or dinitro hydrocarbon, and mixtures thereof; wherein the second solvent used in step-(b) is selected from the group consisting of a ketone, an aliphatic amide, a nitrile, a hydrocarbon, a cyclic ether, an aliphatic ether, a polar aprotic solvent, and mixtures thereof; wherein the third solvent used in step-(c) is selected from the group consisting of a hydrocarbon, a cyclic ether, an aliphatic ether, a chlorinated hydrocarbon, and mixtures thereof; wherein the fourth solvent used in step-(d) is selected from the group consisting of a hydrocarbon, cyclic ethers, an ether, an ester, a nitrile, an aliphatic amide, a chlorinated hydrocarbon, and mixtures thereof; and wherein the fifth solvent used in step-(e) is selected from the group consisting of an alcohol, a hydrocarbon, a cyclic ether, an aliphatic ether, a chlorinated hydrocarbon, and mixtures thereof.

5. The process of claim 4, wherein the first solvent used in step-(a) is selected from the group consisting of n-pentane, n-hexane, n-heptane, cyclohexane, methylene chloride, dichloro ethane, chloroform, carbon tetrachloride, dichlorobenzene, nitrobenzene, dinitrobenzene, and mixtures thereof; wherein the second solvent used in step-(b) is selected from the group consisting of acetone, methyl ethyl ketone, methyl isobutyl ketone, methyl tert-butyl ketone, acetonitrile, tetrahydrofuran, 2-methyl tetrahydrofuran, 1,4-dioxane, diethyl ether, diisopropyl ether, methyl tert-butyl ether, monoglyme, diglyme, n-pentane, n-hexane, n-heptane, cyclohexane, toluene, xylene, N,N-dimethylformamide, N,N-dimethylacetamide, dimethylsulfoxide, N-methylpyrrolidone, and mixtures thereof; wherein the third solvent used in step-(c) is selected from the group consisting of tetrahydrofuran, 2-methyl tetrahydrofuran, 1,4-dioxane, diethyl ether, diisopropyl ether, methyl tert-butyl ether, n-pentane, n-hexane, n-heptane, cyclohexane, toluene, xylene, dichloromethane, dichloroethane, chloroform, and mixtures thereof; wherein the fourth solvent used in step-(d) is selected from the group consisting of tetrahydrofuran, 2-methyl tetrahydrofuran, 1,4-dioxane, diethyl ether, diisopropyl ether, methyl tert-butyl ether, dimethoxyethane, diethoxyethane, n-pentane, n-hexane, n-heptane, cyclohexane, toluene, benzene, xylene, dichloromethane, dichloroethane, chloroform, ethyl acetate, isopropyl acetate, tert-butyl acetate, acetonitrile, propionitrile, N,N-dimethylformamamide, N,N-dimethylacetamide, and mixtures thereof; and wherein the fifth solvent used in step-(e) is selected from the group consisting of methanol, ethanol, isopropyl alcohol, n-propanol, n-butanol, tetrahydrofuran, 2-methyl tetrahydrofuran, 1,4-dioxane, diethyl ether, diisopropyl ether, methyl tert-butyl ether, dimethoxyethane, diethoxyethane, n-pentane, n-hexane, n-heptane, cyclohexane, toluene, xylene, dichloromethane, dichloroethane, chloroform, and mixtures thereof.

6. The process of claim 1, wherein the Lewis acid catalyst used in step-(a) is selected from the group consisting of aluminium chloride, aluminium bromide, zinc chloride, zinc bromide, boron trifluoride, and mixtures thereof; wherein the nitrating agent used in step-(b) is selected from the group consisting of silver nitrite, sodium nitrite, silver chloride and silver nitrate, and mixtures thereof; wherein the metal iodide employed for facilitating the nitration reaction in step-(b) is potassium iodide or sodium iodide; wherein the ester suppressant employed in the step-(b) is benezene-1,3,5-triol; wherein the azodicarboxylate used in step-(d) is selected from the group consisting of a di-(C₁₋₄ alkyl)azodicarboxylate, dibenzyl azodicarboxylate and bis-(2,2,2-trichloroethyl)azodicarboxylate; wherein the reaction in step-(d) is performed in the presence of a phosphine ligand; and wherein the acid used in step-(e) is a mineral acid or an organic acid.

7. The process of claim 6, wherein the Lewis acid catalyst used in step-(a) is aluminium chloride; wherein the nitrating agent used in step-(b) is silver nitrite; wherein the azodicarboxylate used in step-(d) is selected from the group consisting of diethyl azodicarboxylate, diisopropyl azodicarboxylate, di-n-propylazodicarboxylate, di-tert-butyl azodicarboxylate and diisobutyl azodicarboxylate; wherein the phosphine ligand is selected from the group consisting of tributylphosphine, trioctylphosphine, triphenylphosphine and tri (o-tolyl)phosphine; and wherein the acid used in step-(e) is selected from the group consisting of hydrochloric acid, hydrobromic acid, sulfuric acid, acetic acid, propionic acid, butanoic acid, pentanoic acid, hexanoic acid, and mixtures thereof.

8. The process of claim 1, wherein the acylation reaction in step-(a) is carried out at a temperature of about 0° C. to about 100° C. for about 2 hours to about 40 hours; wherein the nitration reaction in step-(b) is carried out at a temperature of about 0° C. to about 50° C. for about 30 minutes to about 7 hours; wherein the reaction in step-(c) is carried out at a temperature of about −5° C. to about 80° C.; wherein the reaction in step-(d) is carried out at a temperature of about −5° C. to about 50° C. for at least 30 minutes; and wherein the reaction in step-(e) is carried out at a temperature of about −5° C. to about 80° C. for at least 30 minutes.

9. The process of claim 8, wherein the acylation reaction in step-(a) is carried out at a temperature of about 20° C. to about 30° C. for about 28 hours to about 32 hours; wherein the nitration reaction in step-(b) is carried out at a temperature of about 20° C. to about 40° C. for about 3 hours to about 5 hours; wherein the reaction in step-(c) is carried out at a temperature of about 15° C. to about 35° C.; wherein the reaction in step-(d) is carried out at a temperature of about 0° C. to about 10° C. for about 2 hours to about 3 hours; and wherein the reaction in step-(e) is carried out at a temperature of about 20° C. to about 40° C. for about 2 hours to about 4 hours.

10. The process of claim 1, wherein the reducing agent used in step-(c) is selected from the group consisting of L-selectride, (−)-β-Chlorodiisopinocampheyl borane, Rutheneium and Rhodium complexes, and a borane complex with dimethyl sulfide, N,N-diethylaniline, tetrahydrofuran, picoline, triethylamine, dimethylamine, pyridine, ter-butylamine, 4-methylmorpholine, N-phenyl-morpholine, N-ethyl-N-isopropylaniline and N,N-diisopropylethylamine; and wherein the chiral auxiliary used in step-(c) is selected from the group consisting of (1S,2S)-cis-1-amino-2-indanol, (R) or (S)-2-methyl-CBS-oxazaborolidine, (R) or (S)-o-tolyl-CBS-oxazaborolidine, (R) or (S)-2-(diphenylhydroxymethyl)pyrrolidine, (1S,2R)-2-amino-1,2-diphenylethanol, (R)-(−)-2-amino-2-phenylethanol, (R)-2-amino-3-methyl-1,1-diphenyl-1-butanol, and (1S,2S)-1-amino-1,2,3,4-tetrahydro-naphthalen-2-ol.

11. The process of claim 10, wherein the reducing agent used in step-(c) is a borane complex with dimethyl sulfide or N,N-diethylaniline; and wherein the chiral auxiliary used in step-(c) is (R) or (S)-2-methyl-CBS-oxazaborolidine.

12. The process of claim 1, wherein the reducing agent used in step-(e) is selected from the group consisting of noble metal catalysts and their compounds, raney-nickel, ferrous sulfate heptahydrate in aqueous ammonia, iron, zinc, cobalt, ferric chloride-hydrazine hydrate, sodium dithionite, tin chloride hydrate, tin chloride hydrate-hydrochloric acid, tin-hydrochloric acid, zinc-ammonium formate, zinc-formic acid, zinc-acetic acid, zinc-hydrochloric acid, zinc-hydrazinium mono formate, magnesium-ammonium formate, and mixtures thereof.

13. The process of claim 12, wherein the reducing agent used in step-(e) is zinc dust.

14. The process of claim 1, wherein the stereochemically isomeric form of the substituted phenylcyclopropylamine derivative of formula II obtained in step-(e) is trans-(1R,2S)-2-(3,4-difluorophenyl)-cyclopropylamine of formula IIa (formula II, wherein R¹, R² and R⁵ are H, and R³ and R⁴ are F):

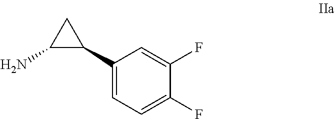 [SMILES: N[C@@H]1C[C@H]1c1ccc(F)c(F)c1]

15. The process of claim 1, wherein the stereochemically isomeric form of the substituted phenylcyclopropylamine derivative of formula II obtained in step-(e) is trans-(1S,2R)-2-(3,4-difluorophenyl)-cyclopropylamine of formula IIb (formula II, wherein R¹, R² and R⁵ are H, and R³ and R⁴ are F):

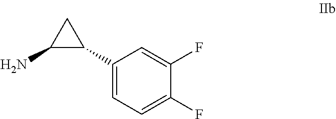 [SMILES: N[C@H]1C[C@@H]1c1ccc(F)c(F)c1]

16-22. (canceled)

23. A process for preparing ticagrelor or a pharmaceutically acceptable salt thereof from a substituted phenylcyclopropylamine derivative of formula II: 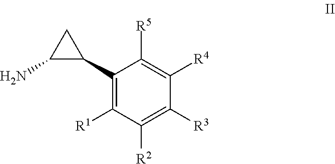
or a stereochemically isomeric form or a mixture of stereochemically isomeric forms thereof, or an acid addition salt thereof; wherein R¹, R², R³, R⁴ and R⁵ are, each independently, selected from hydrogen and a halogen atom, with the proviso that the benzene ring is substituted with at least one or more halogen atoms, wherein the halogen atom is F, Cl, Br or I; wherein the substituted phenylcyclopropylamine derivative of formula II is formed by a method comprising:
b) reacting a halogen substituted phenyl compound of formula VII: 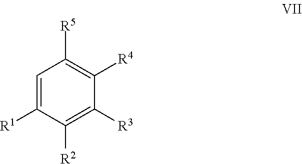
wherein R¹, R², R³, R⁴ and R⁵ are as defined in formula II; with a 3-chloropropionyl halide compound of formula VIII: 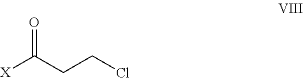
wherein ‘X’ is a leaving group, selected from the group consisting of hydroxy, Cl, Br and I; in the presence of a Lewis acid in a first solvent to produce an acylated compound of formula VI: 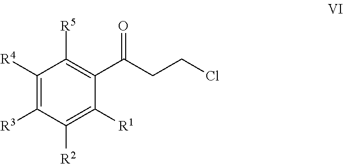
wherein R¹, R², R³, R⁴ and R⁵ are as defined above;
b) nitrating the compound of formula VI with a nitrating agent, in the presence or absence of a metal iodide and an ester suppressant, in a second solvent to produce a substituted 3-nitro-1-propanone compound of formula V: 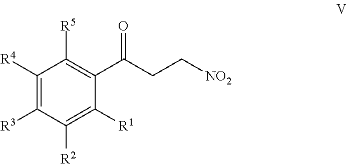
c) subjecting the compound of formula V to asymmetric reduction with a reducing agent in the presence of a chiral auxiliary in a third solvent to produce an optically active substituted 3-nitro-1-propanol compound of formula IV: 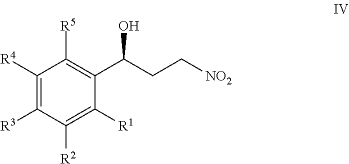
or a stereochemically isomeric form thereof;
d) subjecting the compound of formula IV to intramolecular cyclization in the presence of an azodicarboxylate, optionally in the presence of a phosphine ligand, in a fourth solvent to produce an optically active substituted nitrocyclopropane compound of formula III: 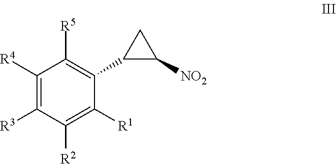
or a stereochemically isomeric form thereof or a mixture of stereochemically isomeric forms thereof; and
reducing the substituted nitrocyclopropane compound of formula III with a reducing agent, optionally in the presence of an acid, in a fifth solvent to produce the substituted phenylcyclopropylamine derivatives of formula II or a stereochemically isomeric form or a mixture of stereochemically isomeric forms thereof, and optionally converting the compound of formula II obtained into an acid addition salt thereof.
